(19) United States
(12) Patent Application Publication
(10) Pub. No.: US 2013/0156773 A1
(43) Pub. Date: Jun. 20, 2013

(54) LUNG REGENERATION USING CORD BLOOD-DERIVED HEMATOPOIETIC STEM CELLS

(71) Applicant: Monique E. Depaepe, Barrington, RI (US)

(72) Inventor: Monique E. Depaepe, Barrington, RI (US)

(73) Assignee: WOMEN AND INFANTS HOSPITAL OF RHODE ISLAND, Providence, RI (US)

(21) Appl. No.: 13/805,003

(22) PCT Filed: Jun. 17, 2011
(86) PCT No.: PCT/US11/40826

§ 371 Date: Mar. 4, 2013

Related U.S. Application Data
(60) Provisional application No. 61/356,156, filed on Jun. 18, 2010.
(60) Provisional application No. 61/438,481, filed on Feb. 1, 2011.

(51) Int. Cl. A61K 35/14

(52) U.S. Cl. CPC A61K 35/14
USPC 424/136.1; 424/93.7

(57) ABSTRACT

Described herein are novel approaches for regenerating injured or defective lung epithelium for the treatment of respiratory disorders using, in part, cord blood-derived hematopoietic stem cells. The methods and uses described herein relate to the administration of or use of hematopoietic stem cells, specifically those isolated or enriched from umbilical cord blood, to a subject in need thereof having a respiratory disorder, such as BPD, or any disorder characterized by insufficient or defective or injured lung epithelium or lung vasculature.

### CROSS-REFERENCE TO RELATED APPLICATIONS

This application claims benefit under 35 U.S.C. §119(e) of U.S. Provisional Application Ser. No. 61/438,481 filed on Feb. 1, 2011 and U.S. Provisional Application Ser. No. 61/356,156 filed on Jun. 18, 2010, the contents of each of which are herein incorporated by reference in their entireties.

### GOVERNMENT SUPPORT

This invention was made with Government support under NIII P20-RR18728 and NIH-p-20-RR018757 COBRE awarded by the National Institutes of Health (NIH). The Government has certain rights in the invention.

### FIELD OF THE INVENTION

The present invention relates to novel methods for the treatment of respiratory disease using cord blood-derived hematopoietic stem cells.

### BACKGROUND OF THE INVENTION

Each year, 5000-10,000 newborns and premature infants suffer from bronchopulmonary dysplasia (BPD), a chronic lung disease that follows ventilator and oxygen therapy for acute respiratory failure after premature birth, and is associated with significant mortality and long-term morbidity. An estimated 30% of infants with a birth weight between 500 and 1,500 g will develop BPD. Many of these infants require long-term ventilation and/or supplemental oxygen. The main pathological hallmark of BPD is an arrest of alveolar development, characterized by large and simplified distal airspaces that show little evidence of vascularized ridges (secondary crests) or alveolar septa. In addition, the lungs of infants with BPD show structurally abnormal microvasculature and variable degrees of interstitial fibrosis. Further, several recent reports have shown that the lungs of ventilated preterm infants with early BPD show markedly increased levels of alveolar epithelial cell death, and it was recently demonstrated that increased alveolar epithelial apoptosis in newborn mice is sufficient to disrupt alveolar remodeling (De Paepe, Am J Pathol. 2008 July; 173(1):42-56). Many risk factors have been implicated in the pathogenesis of BPD, but among these, prematurity, oxygen toxicity, and barotrauma are considered central to a final common outcome. In addition, there are variable contributions of infection/inflammation, glucocorticoid exposure, chorioamnionitis, and genetic polymorphisms. The precise mechanisms whereby these predisposing conditions result in disrupted alveolar development remain primarily unknown.

Emphysema, defined as airspace enlargement distal to terminal bronchioles, is a major component of chronic obstructive pulmonary disease (COPD), the fourth leading cause of death in the US. BPD and emphysema are characterized by interrupted development and loss of alveolar structures, and therapy is palliative. Other lung diseases that currently lack specific treatments and involve damage to respiratory and pulmonary structures and or function include other causes of COPD, Cystic Fibrosis, fibrosis, Acute Respiratory Distress Syndrome (ARDS), pulmonary hypoplasia and pulmonary hypertension.

### SUMMARY OF THE INVENTION

The inventor has discovered novel approaches for regenerating injured or defective lung epithelium for the treatment of respiratory disorders using, in part, cord blood-derived hematopoietic stem cells. The methods and uses described herein relate to the administration of or use of hematopoietic stem cells, specifically those isolated or enriched from umbilical cord blood, to a subject in need thereof having a respiratory disorder, such as BPD, or any disorder characterized by insufficient or defective or injured lung epithelium or lung vasculature.

Accordingly, in one aspect, provided herein are methods for treating or preventing a lung disorder in a subject in need thereof. In one such aspect, the method comprises administering a therapeutically effective amount of a population of isolated or enriched umbilical cord blood derived hematopoietic stem cells to a subject in need thereof. In some embodiments of this aspect, the method further comprises selecting a subject who is suffering from a lung disorder prior to administering the population of isolated or enriched umbilical cord blood derived hematopoietic stem cells.

In another aspect, methods for repairing or reconstituting or generating pulmonary epithelium in a subject in need thereof are provided. In one such aspect, the method comprises administering a population of isolated or enriched umbilical cord blood derived hematopoietic stem cells to a subject in need thereof. In some embodiments of this aspect, the method further comprises selecting a subject in need of repair or reconstitution or generation of pulmonary epithelium prior to administering the population of isolated or enriched umbilical cord blood derived hematopoietic stem cells.

In one aspect, methods for repairing or reconstituting or generating pulmonary vasculature or pulmonary endothelium are provided. In one such aspect, the method comprises administering a population of isolated or enriched umbilical cord blood derived hematopoietic stem cells to a subject in need thereof. In some embodiments, the method further comprises selecting a subject in need of repair or reconstitution or generation of pulmonary vasculature or pulmonary endothelium prior to administering the population of isolated or enriched umbilical cord blood derived hematopoietic stem cells.

In another aspect, methods are provided for repairing or reconstituting pulmonary alveoli in a subject. In one such aspect, the method comprises administering a population of isolated or enriched umbilical cord blood derived hematopoietic stem cells to a subject in need thereof. In some embodiments of this aspect, the method further comprises selecting a subject in need of repair or reconstitution or regeneration of pulmonary alveoli prior to administering the population of isolated or enriched umbilical cord blood derived hematopoietic stem cells.

In some embodiments of these aspects and all such aspects described herein, the administration of the population of isolated or enriched umbilical cord blood derived hematopoietic stem cells is via intrapulmonary administration, systemic administration, or a combination thereof. In some such embodiments, the intrapulmonary administration is intratreacheal or intranasal administration.

In another aspect, methods are provided for repairing or reconstituting pulmonary epithelium. In one such aspect, the method comprises intrapulmonary administration of a population of isolated or enriched umbilical cord blood derived hematopoietic stem cells to a subject in need thereof.

In one aspect, methods for repairing or reconstituting pulmonary alveoli in a subject are provided, where the method comprises administering a population of isolated or enriched umbilical cord blood derived hematopoietic stem cells to a subject, where the administration is intrapulmonary, systemic, or a combination thereof.

In another aspect, methods for treating an infant or preterm subject suffering from bronchopulmonary dysplasia are provided. In one such aspect, the method comprises administering a population of isolated or enriched umbilical cord blood derived hematopoietic stem cells to an infant or preterm subject suffering from bronchopulmonary dysplasia, where the administration is intrapulmonary, systemic, or a combination thereof.

In some embodiments of these aspects and all such aspects described herein, the isolated or enriched umbilical cord blood derived hematopoietic stem cells are expanded ex vivo prior to administration to the subject. In some embodiments of the aspects described herein, the hematopoietic stem cells are selected based on positive expression of the cell-surface molecule CD34.

In some embodiments of these aspects and all such aspects described herein, the subject is an intubated subject. In some embodiments of these aspects and all such aspects described herein, the subject is an infant or preterm infant.

In some embodiments of these aspects and all such aspects described herein, the population of isolated or enriched umbilical cord blood derived hematopoietic stem cells are autologous cells. In other embodiments of these aspects and all such aspects described herein, the population of isolated or enriched umbilical cord blood derived hematopoietic stem cells are allogeneic cells obtained from one or more donors. In some embodiments of thes aspects and all such aspects described herein, the population of isolated or enriched umbilical cord blood derived hematopoietic stem cells comprise autologous cells and allogeneic cells obtained from one or more donors.

In some embodiments of these aspects and all such aspects described herein, the methods further or also comprise administering at least one therapeutic agent with the population of isolated or enriched umbilical cord blood derived hematopoietic stem cells. In some embodiments of these aspects and all such aspects described herein, the methods further comprise arming the population of isolated or enriched umbilical cord blood derived hematopoietic stem cells with at least one therapeutic agent. In some such embodiments, the at least one therapeutic agent enhances homing, engraftment, or survival of the population of isolated or enriched umbilical cord blood derived hematopoietic stem cells. In some embodiments, the at least one therapeutic agent comprises a bispecific antibody. In some embodiments, the bispecific antibody is an antibody specific for a hematopoietic stem cell marker and a cell-surface protein that mediates the adhesion of lymphocytes, monocytes, eosinophils, and basophils to vascular endothelium. In some such embodiments, the hematopoietic stem cell marker is CD34. In some embodiments, the cell-surface protein that mediates the adhesion of lymphocytes, monocytes, eosinophils, and basophils to vascular endothelium is VCAM-1. In some embodiments, the bispecific antibody is specific for CD34 and VCAM-1.

In other aspects, provided herein are populations of isolated or enriched umbilical cord blood-derived hematopoietic stem cells for use in treating or preventing a lung disorder.

In some aspects, provided herein are populations of isolated or enriched umbilical cord blood-derived hematopoietic stem cells for use in repairing, reconstituting, or generating pulmonary epithelium.

In other aspects, provided herein are populations of isolated or enriched umbilical cord blood-derived hematopoietic stem cells for use in repairing, reconstituting, or generating pulmonary vasculature or pulmonary endothelium.

In some aspects, provided herein are populations of isolated or enriched umbilical cord blood-derived hematopoietic stem cells for use in repairing or reconstituting pulmonary alveoli

In some embodiments of these aspects and all such aspects described herein, the populations of isolated or enriched umbilical cord blood-derived hematopoietic stem cells are administered via intrapulmonary administration, systemic administration, or a combination thereof. In some such embodiments, the intrapulmonary administration is intratreacheal or intranasal administration.

In other aspects, provided herein are populations of isolated or enriched umbilical cord blood derived hematopoietic stem cells for use in repairing or reconstituting pulmonary epithelium by intrapulmonary administration.

In some aspects, provided herein are populations of isolated or enriched umbilical cord blood derived hematopoietic stem cells for use in repairing or reconstituting pulmonary alveoli by intrapulmonary administration, systemic administration, or a combination thereof.

In other aspects, provided herein are populations of isolated or enriched umbilical cord blood derived hematopoietic stem cells for use in treating bronchopulmonary dysplasia in an infant or preterm subject by intrapulmonary administration, systemic administration, or a combination thereof.

In some embodiments of these aspects and all such aspects described herein, the populations of isolated or enriched umbilical cord blood derived hematopoietic stem cells are first expanded ex vivo.

In some embodiments of these aspects and all such aspects described herein, the hematopoietic stem cells are selected based on positive expression of CD34.

In some embodiments of these aspects and all such aspects described herein, the population of isolated or enriched umbilical cord blood derived hematopoietic stem cells comprise autologous cells.

In some embodiments of these aspects and all such aspects described herein, the population of isolated or enriched umbilical cord blood derived hematopoietic stem cells comprise allogeneic cells obtained from one or more donors.

In some embodiments of these aspects and all such aspects described herein, the uses further comprise administering at least one therapeutic agent. In some embodiments of these aspects and all such aspects described herein, the uses further comprise arming the population of isolated or enriched umbilical cord blood derived hematopoietic stem cells with at least one therapeutic agent. In some such embodiments the at least one therapeutic agent enhances homing, engraftment, or survival of the the population of isolated or enriched umbilical cord blood derived hematopoietic stem cells. In some such embodiments, the at least one therapeutic agent comprises a bispecific antibody. In some embodiments, the bispecific antibody is an antibody specific for a hematopoietic stem cell marker and a cell-surface protein that mediates the adhesion of lymphocytes, monocytes, eosinophils, and basophils to vascular endothelium. In some such embodiments, the hematopoietic stem cell marker is CD34. In some embodiments, the cell-surface protein that mediates the adhesion of lymphocytes, monocytes, eosinophils, and basophils to vascular endothelium is VCAM-1. In some embodiments, the bispecific antibody is specific for CD34 and VCAM-1.

### BRIEF DESCRIPTION OF THE FIGURES

FIGS. 1A-1B show the morphology of human cord blood-derived CD34⁺ cells in culture. FIG. 1A shows the appearance of CD34⁺ cells after two-week culture in StemPro-34 SFM medium supplemented with SCF, IL-3 and GM-CSF. Cells were mainly round, relatively small and non-adherent, similar to the appearance of freshly isolated CD34+ cells. FIG. 1B shows the appearance of CD34⁺ cells after two-week culture in modified MTEC medium. The majority of cells were adherent with cell shapes ranging from round to elongated with prominent cellular extensions.

FIG. 2 shows an RT-PCR analysis of respiratory epithelial gene expression in cultured CD34⁺ cells. Cells were exposed to various culture media, growth factors and cytokines aimed at inducing respiratory epithelial differentiation. Shown are the results of one representative isolate. Expression of TTF-1 is seen in most conditions. Expression of SP-C and CFTR is seen in the presence of dexamethasone (DEX) and in MTEC medium. Sporadic expression of AQP5 is seen in the presence of MTEC medium. CCSP expression is not seen. Positive (human lung) and negative controls (omission reverse transcriptase, H₂O control) were included. The housekeeping gene GAPDH was included as loading control.

FIG. 3 shows an analysis of alveolar development in DOX-treated single transgenic CCSP+/FasL− and double transgenic CCSP+/FasL+ mice at postnatal week 8. The mean cord length at 8 weeks was significantly larger in double transgenic mice compared with single transgenic animals (P<0.02), indicative of disrupted alveolar development, mimicking the alveolar pathology of BPD.

FIGS. 4A-4B show an analysis of homing of intranasally delivered hUCB-CD34+ cells to distal lung parenchyma. FIGS. 4A and 4B show representative anti-human vimentin staining of murine lungs on post-transplantation day 2, showing diffuse presence of human cells in the distal airways and airspaces. FIGS. 4A and 4B show anti-human vimentin staining, using avidin-biotin-peroxidase. Original magnification ×100 in FIG. 4A and ×400 in FIG. 4B.

FIG. 5 demonstrates engraftment of hUCB-CD34+ cells using anti-human vimentin immunohistochemistry. Several human vimentin immunoreactive cells are noted within the alveolar septa 8 weeks post-transplantation, confirming successful long-term engraftment of hUCB-CD34+-derived cells. Anti-human vimentin staining was done using avidin-biotin-peroxidase. Original magnification used was ×200.

FIGS. 6A-6B demonstrates engraftment of hUCB-CD34+ cells by FISH analysis using human chromosome-specific centromeric probes (bright dots on shaded cells). FIG. 6A shows data from post-transplantation day 2 and that several FISH-positive human cells are present within the alveolar lumen. FIG. 6B shows data from post-transplantation week 8. Human-derived FISH-positive cell is noted incorporated in the alveolar septum. FISH analysis was performed using FITC-labeled centromeric enumeration probes complementary to human chromosomes X, Y and 18.

FIG. 7 demonstrates epithelial differentiation of engrafted hUCB-CD34+ cells using cytokeratin. Anti-human cytokeratin staining at post-transplantation week 8 shows several cytokeratin-positive, human-derived epithelial cells within the alveolar septa. Two cells with morphologic appearance of alveolar type II cells are noted in close proximity to each other. Anti-human cytokeratin staining is shown using avidin-biotin-peroxidase and an original magnification of ×200.

FIG. 8 demonstrates alveolar type I cell differentiation of engrafted hUCB-CD34+ cells using a double transgenic mouse, post-transplantation week 8. Combined anti-human cytokeratin (Alexafluor 488) and anti-human/mouse T1α (Cy3) immunofluorescence using confocal microscopy is shown. The figure shows stable engraftment of a human cytokeratin-positive cell deep within the alveolar wall, surrounded by type I cells.

FIGS. 9A-9C show analyses of the effect of intranasally administered hUCB-CD34+ cells on somatic growth, lung growth, and alveolar development. FIG. 9A shows that intranasally administered CD34+ cells have no effect on body weight. FIG. 9B shows that V(ae) is less in double transgenic than single transgenic animals. However, within each genotype CD34+ cell administration had no effect on lung growth. FIG. 9C shows that alveolar development as measured by mean cord length is greater in double transgenic than single transgenic animals. However, within each genotype CD34+ cell administration had no effect.

FIGS. 10A-10C show analyses of the effect of systemically administered hUCB-CD34+ cells on somatic growth, lung growth, and alveolar development. FIG. 10A shows that intraperitoneally administered CD34+ cells have no effect on body weight. FIG. 10B shows that intraperitoneally administered CD34+ cells tend to promote lung growth, both in single and double transgenic animals. FIG. 10C shows that intraperitoneally administered CD34+ cells tend to promote alveolar remodeling in double transgenic animals, resulting in decreased MCL.

FIGS. 11A-11C show analyses of the effect of intraperitoneally administered hUCB-CD34+ cells armed with hCD34×mVCAM-1 bispecific antibodies on somatic growth, lung growth, and alveolar development. FIG. 11A shows that intraperitoneally administered CD34+ cells armed with bispecific antibodies promote somatic growth. FIG. 11B shows that intraperitoneally administered CD34+ cells tend to promote lung growth, both in single and double transgenic animals. Bispecific antibodies have no added effect. FIG. 11C shows that bispecific antibody-armed intraperitoneally administered CD34+ cells significantly promote alveolar remodeling in double transgenic animals, resulting in MCL equivalent to that of single transgenic littermates. This data indicates that systemic delivery of hUCB-CD34+ cells, when targeted to the pulmonary microvasculature, can prevent/treat the alveolar disruption characteristic of BPD.

FIGS. 12A-12F demonstrate analysis of engraftment of intranasally delivered CB-CD34+ cells. FIG. 12A shows post-inoculation day 2 (postnatal day 7, P7), double-transgenic recipient. Representative photomicrograph showing scattered mononuclear cells, consistent with CB-CD34+ cells, in the airspaces. A mixed inflammatory aggregate associated with degenerating mononuclear cells is noted in the right lower corner. (Hematoxylin-eosin staining). FIG. 12B shows post-inoculation day 2 (P7), double-transgenic recipient. Representative anti-human vimentin staining showing diffuse distribution of human cord blood-derived cells in the distal airways and airspaces. Murine mesenchymal cells such as fibroblasts, endothelial cells, and peribronchial/perivascular smooth muscle cells showed no cross-reactivity with anti-human vimentin antibody, supporting its specificity for human cells. (Avidin-biotin peroxidase staining, hematoxylin counterstain) FIG. 12C shows a post-inoculation week 8, single-transgenic recipient. Human cord blood-derived cell (from male donor), labeled with coded probes complementary to human chromosomes 18, X and Y, is noted incorporated in the alveolar septum (arrow). (FISH analysis using human chromosome-specific centromeric probes, DAPI counterstain) FIG. 12D shows Alu FISH analysis of human postmortem lung tissue (positive control) showing nuclear positivity in all cells. FIG. 12E shows a post-inoculation week 8, double-transgenic recipient. Five cord blood-derived alu FISH-positive cells are shown along alveolar septum. Four cells occur as doublets (right), indicative of recent replication. FIG. 12F shows a post-inoculation week 8, double-transgenic recipient. Three contiguous alu FISH-positive cells are noted along alveolar septum, suggestive of clonal derivation from a common cord blood-derived precursor. An additional alu-FISH-positive cell is present on the right. (12D-12F: FISH analysis using human alu-specific probes, DAPI counterstain)

FIGS. 13A-13B depict real-time PCR analysis of human alu sequences in murine lung lysates at 8 weeks post-inoculation. FIG. 13A shows an Alu DNA Index (amount of alu-amplified DNA in CB-CD34+ recipient lungs relative to that detected in PBS-treated, non-transplanted lungs). FIG. 13B shows fraction of Alu DNA (percentage of human DNA relative to total lung DNA content). Values represent mean±SD of at least 3 animals per group. Abbreviations: STG: single transgenic; DTG: double transgenic

FIGS. 14A-14B demonstrate analysis of epithelial differentiation of engrafted CB-CD34+ cells by human cytokeratin immunohistochemistry at 8 weeks post-inoculation. FIG. 14A shows a single transgenic recipient. Representative staining result showing a large-sized, ovoid, cord blood-derived, cytokeratin-positive epithelial cell in the alveolar wall. FIG. 14B shows a Double transgenic recipient. Several human-derived, cytokeratin-positive epithelial cells are noted within the alveolar septa. Two large-sized, spherical cells with morphologic appearance of alveolar type II cells are noted in close proximity to each other. Murine lung epithelial cells are not stained, confirming the species-specificity of the anti-human cytokeratin antibody. (Avidin-biotin-peroxidase, hematoxylin counterstain. Original magnification: ×600)

FIG. 15 shows fractions of SP-C immunoreactive cord blood-derived epithelial cells at 8 weeks post-inoculation. Values represent mean±SD of at least 3 animals per group, expressed as a percentage. *: P<0.01. Abbreviations: STG: single transgenic; DTG: double transgenic.

FIG. 16 demonstrates proliferative activity of engrafted cord blood-derived cells at 8 weeks post-inoculation. Fraction of Ki67-positive murine nuclei (“mouse”) and alu FISH-positive cord blood-derived nuclei (“human”), expressed as a percentage. Values represent mean±SD of at least 3 animals per group. *: P<0.05; **: P<0.01 versus murine cells; ***: P<0.05 versus single transgenic animals. Abbreviations: STG: single transgenic; DTG: double transgenic

FIGS. 17A-17O demonstrate analysis of respiratory epithelial differentiation of engrafted CB-CD34+ cells at 8 weeks post-inoculation. FIGS. 17A-17C show a single transgenic recipient. Combined anti-human cytokeratin and anti-mouse/human surfactant protein-C (SP-C) immunofluorescence staining showing one of rare SP-C positive human derived epithelial cells detected in lungs of single transgenic animals. (Anti-human cytokeratin staining combined with anti-SP-C staining, DAPI counterstain). FIGS. 17D-17F show a double transgenic recipient. Colocalization of immunoreactive human cytokeratin and SP-C in cord blood-derived type II-like epithelial cell within the alveolar wall. Arrows indicate the presence of surfactant and human cytokeratin immunoreactive material in adjacent elongated cells, suggestive of intermediate cells generated during transition from type II cells to type I cells. A resident murine type II cell is noted in the left upper corner. (Anti-human cytokeratin staining combined with anti-SP-C staining, DAPI counterstain). FIGS. 17G-17I show a double transgenic recipient. Other example of a human cord-blood derived type II-like cell, characterized by the large size, ovoid shape and presence of abundant human cytokeratin- and SP-C-immunoreactive material in the cytoplasm. Granular surfactant staining, consistent with lamellar bodies, is noted in juxtamembranous location, suggestive of secretory activity (exocytosis) (arrow head). Adjacent cells with elongated cell shape contain surfactant immunoreactive material as well as human cytokeratin, consistent with transitional type II-type I cells (arrows). (Anti-human cytokeratin staining combined with anti-SP-C staining, DAPI counterstain). FIGS. 17J-17L show a double transgenic recipient. Human cord blood-derived surfactant-producing type II-like epithelial cell undergoing mitosis. (Anti-human cytokeratin staining combined with anti-SP-C staining, DAPI counterstain) FIGS. 17M-17O show a double transgenic recipient. Cellular colocalization of T1alpha and human cytokeratin is noted in attenuated cells adjacent to human cord blood-derived type II like cells (arrows), indicative of cord blood-derived type I cells. The cord blood-derived type II-like cell is incorporated deeply within the alveolar wall and partially covered by type I cell extensions. (Anti-human cytokeratin staining combined with anti-T1 alpha staining, DAPI counterstain). Size bar=10 μm

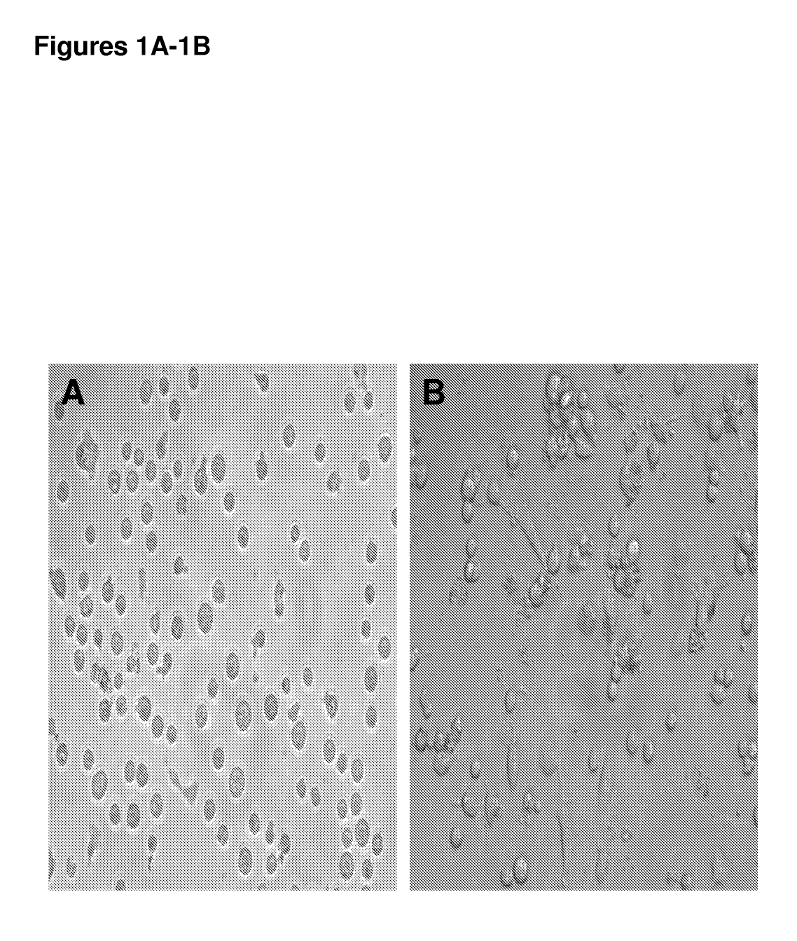

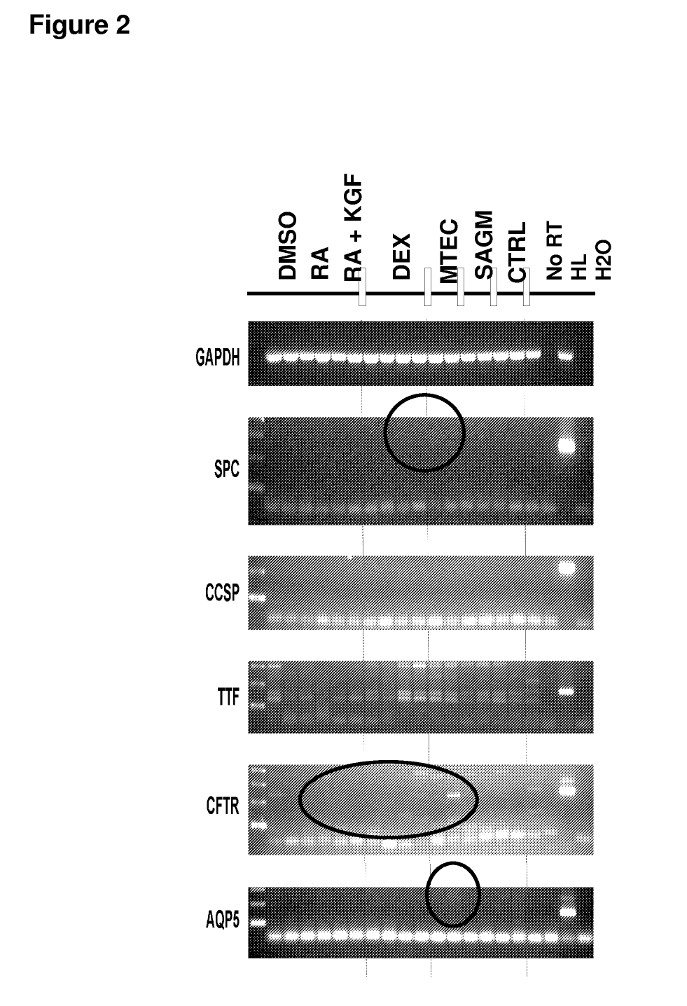

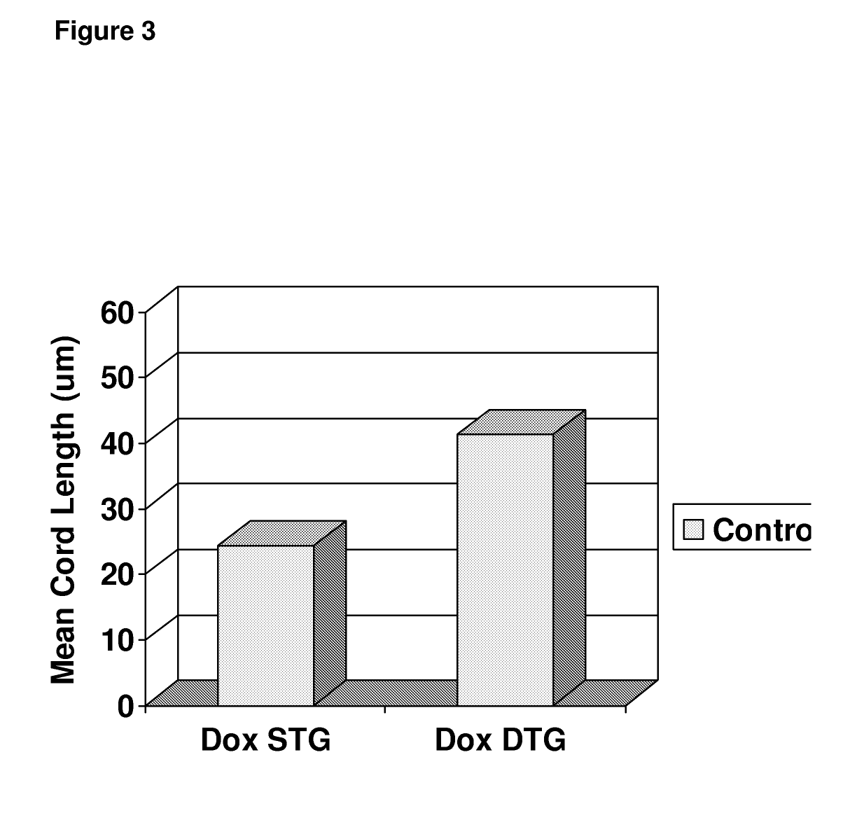

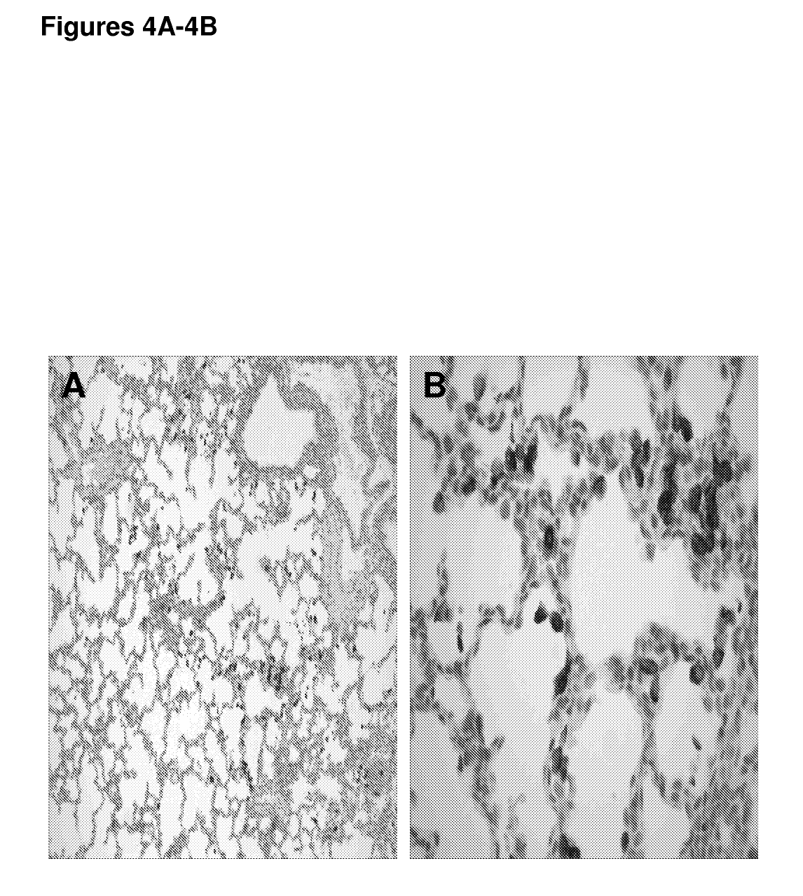

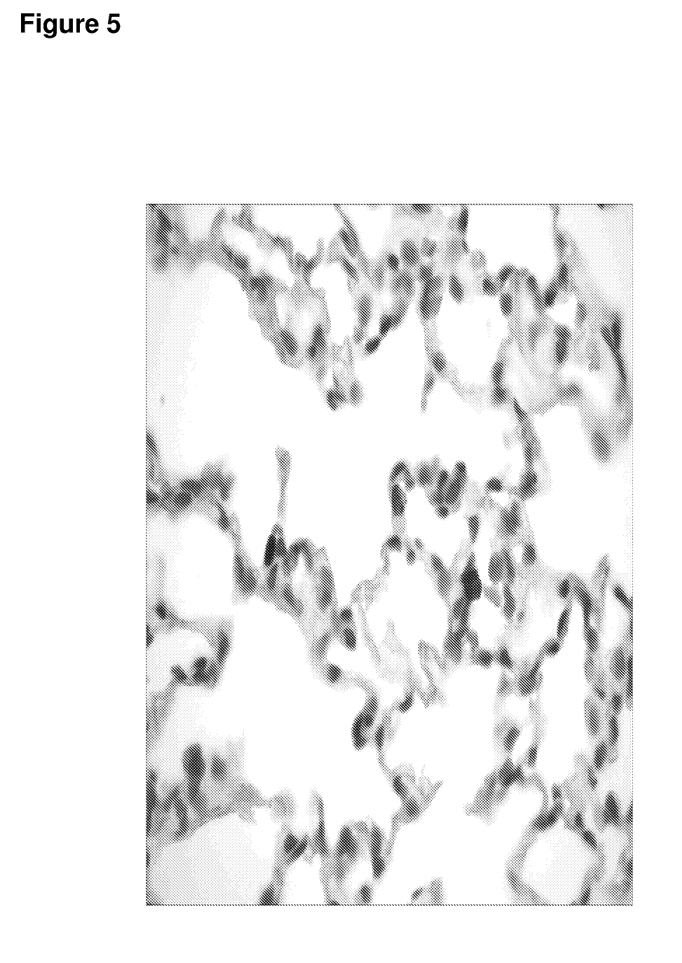

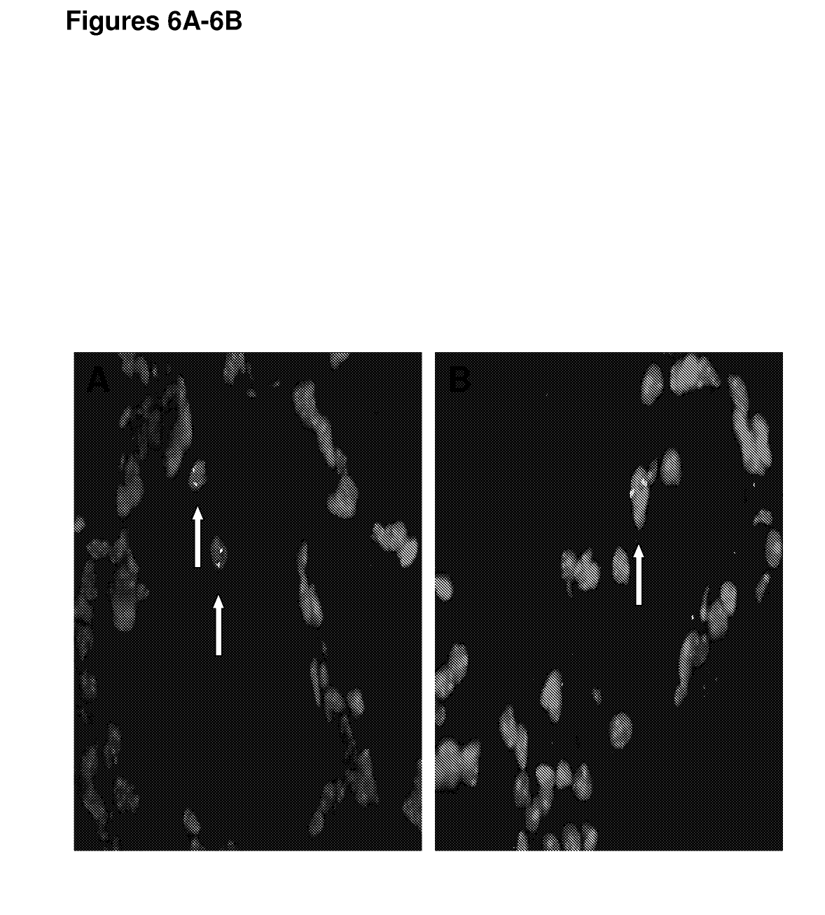

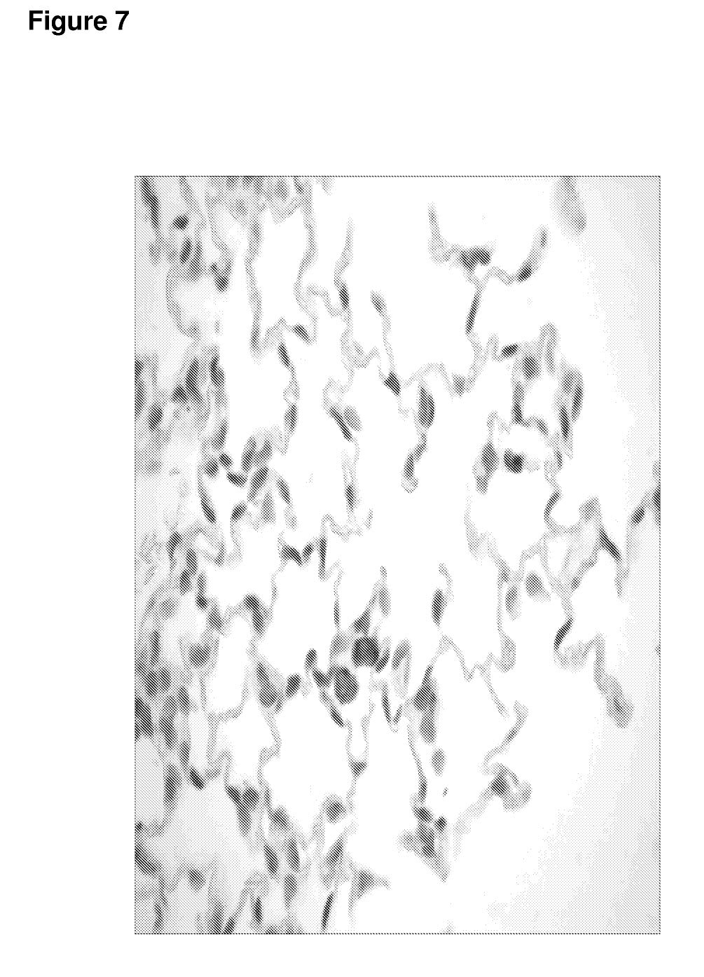

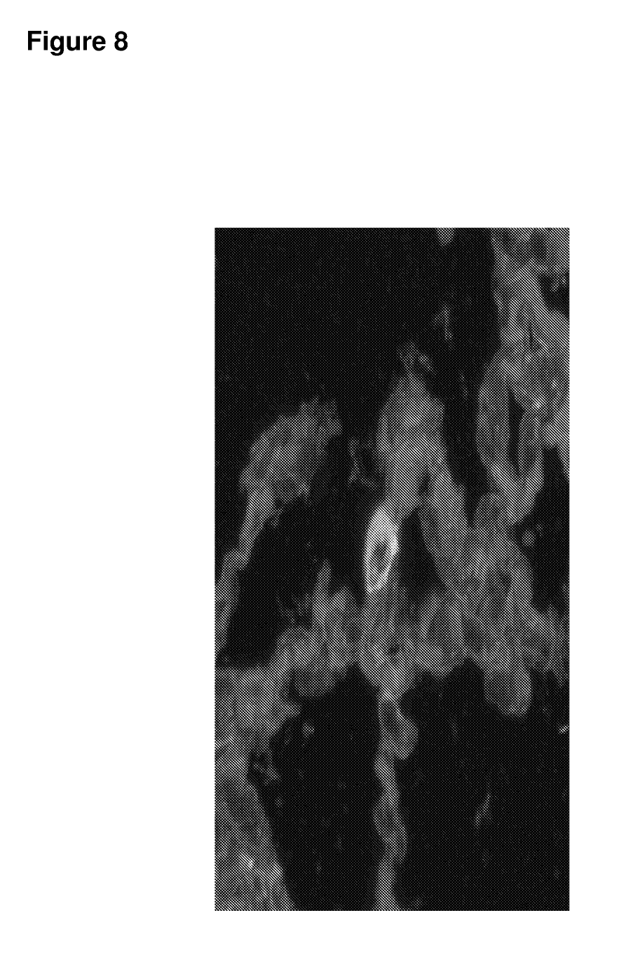

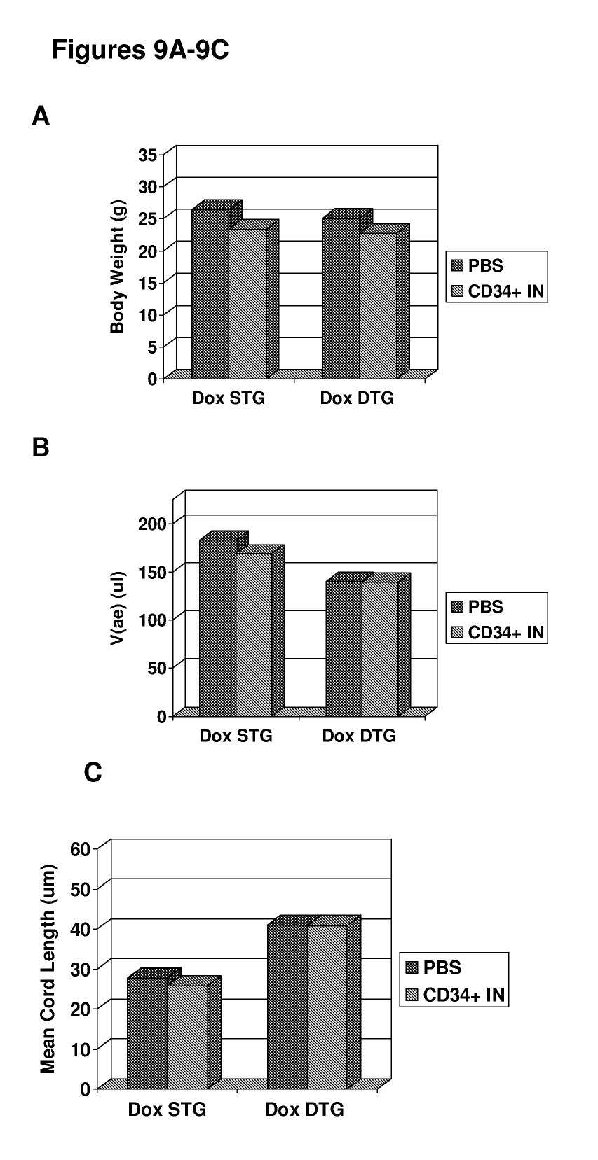

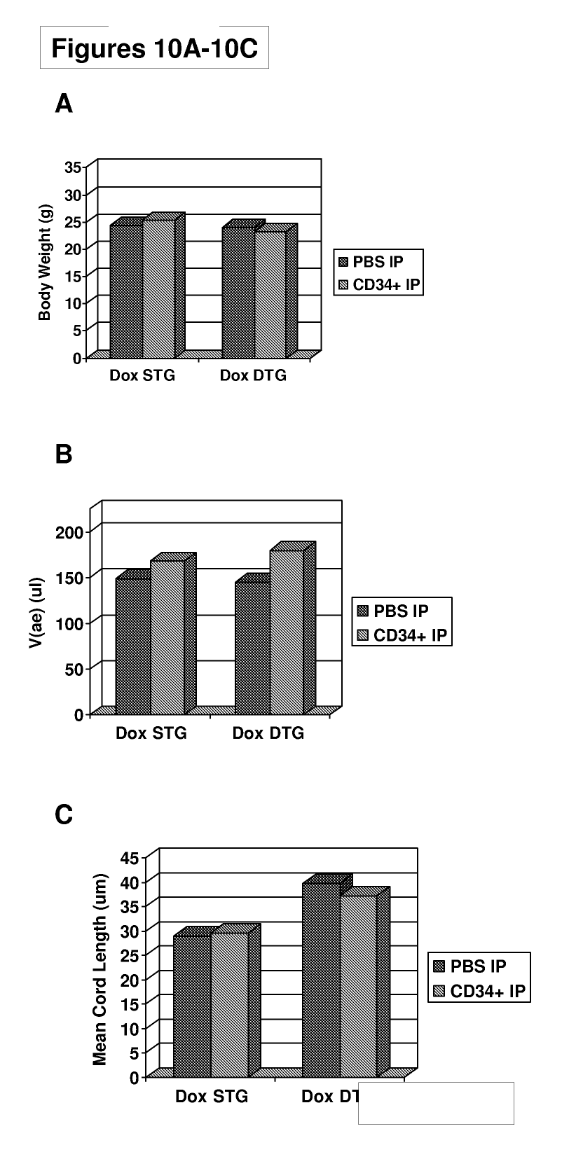

### DETAILED DESCRIPTION

The invention described herein generally relates to the discovery of new and enhanced methods for repairing pulmonary tissue, such as respiratory epithelial cells, and respiratory vasculature, and provides compositions, methods, and uses for treating various lung diseases and conditions using umbilical cord blood-derived hematopoietic stem cells (HSCs), in the absence of other cell populations, such as mesenchymal stem cells. The inventor has discovered, in part, that both source and the route of administration of stem cells for use in such regenerative medicine treatments are important determinants of long-term engraftment and repair of pulmonary tissues and structures.

Specifically, the inventor has discovered, in part, that direct administration to the lungs and airways of a population of hematopoietic stem cells isolated from umbilical cord blood, such as a CD34+ hematopoietic stem cell population, results in long-term engraftment of the administered cells, differentiation into respiratory epithelium, and consequent lung growth, alveolar regeneration, and repair.

Further, the inventor found that administration of such hematopoietic stem cells isolated from umbilical cord blood via a systemic route, such as an intraperitoneal route, enhances pulmonary vascular regeneration and alveolar development. The inventor also discovered, as described herein, that arming of such stem cells with a bispecific antibody targeting the hematopoietic stem cells and a target tissue or antigen, such as, for example, VCAM-1, serves as a therapeutic agent that can enhance homing of the hematopoietic stem cells to the target tissue upon systemic administration, for example, intraperitoneal administration.

Accordingly, provided herein, in part, are methods for the treatment and prevention of a respiratory disease or disorder in a subject in need thereof.

Adult stem cell transplantation has recently emerged as a new alternative to stimulate repair of injured tissues and organs. In the past decade, some studies in animals and humans have documented the ability of adult bone marrow—derived stem cells, i.e., hematopoietic stem cells, to differentiate into an expanding repertoire of nonhematopoietic cell types, including brain, skeletal muscle, chondrocytes, liver, endothelium, and heart. However, the lung and associated respiratory structures have remained relatively resistant to such therapeutic modalities, specifically the use of hematopoietic stem cells alone. There are, however, reports indicating that mesenchymal stem cells can be used for stem cell therapies in the lung, and that hematopoietic stem cells can be co-administered with mesenchymal stem cells in pulmonary transplantation. For example, it has been described that co-transplantation of mesenchymal cells, isolated as non-hematopoietic cells from fetal lung CD34+ cells, enhanced the engraftment of hematopoietic stem cells (Noort et al., Exp Hematol 2002; 30:870-78).

Similarly, it has been described that the repopulation of mice with limited numbers of hematopoietic stem cells, augmented by co-infusion with unrelated human mesenchymal stem cells (Maitra et al., Bone Marrow Transplant. 2004 March; 33(6):597-604). Human mesenchymal stem cells culture has also been shown to support the ex vivo propagation of CD34+ cells, in the absence of direct contact between the mesenchymal and hematopoietic cells in culture, and enhance transplantation. (Sumner, et al., Cytother 2001 3; 422a).

Several other reports also describe the use of mesenchymal stem cells and non-hematopoietic stem cells derived from bone-marrow populations in lung therapies in animal models (Krause DS et al., “Multi-organ, multi-lineage engraftment bya single bone marrow-derived stem cell.” Cell 2001; 105:369-377; Kotton D N, et al. “Bone marrow-derived cells as progenitors of lung alveolar epithelium.” Development 2001; 128:5181-5188; Ortiz L A, et al. “Mesenchymal stem cell engraftment in lung is enhanced in response to bleomycin exposure and ameliorates its fibrotic effects.”Proc Natl Acad Sci USA 2003; 100:8407-8411; Theise N D et al. “Radiation pneumonitis in mice:a severe injury model for pneumocyte engraftment from bone marrow.” Exp Hematol 2002; 30:1333-1338; Abe S et al. “Transplanted BM and BM side population cells contribute progeny to the lung and liver in irradiated mice.” Cytotherapy 2003; 5:523-533; Aliotta J M et al. Bone marrow production of lung cells: the impact of G-CSF, cardiotoxin, graded doses of irradiation, and subpopulation phenotype.” Exp Hematol 2006; 34:230-241.Rojas M et al. “Bone marrow-derived mesenchymal stem cells in repair of the injured lung.” Am J Respir Cell Mol Biol 2005; 33:145-152; Gupta N et al. “Intrapulmonary delivery of bone marrow-derived mesenchymal stem cells improves survival and attenuates endotoxin-induced acute lung injury in mice.” J Immunol 2007; 179:1855-1863; US Patent Application 20090274665, “Stem Cells for Treating Lung Diseases.” Akabutu and Thebeau).

While evidence exists supporting the ability of some types of bone marrow-derived stem cells, i.e., mesenchymal stem cells, to give rise to lung tissue, other reports have been unable to detect significant regeneration of lung tissue with bone marrow cells (Kotton D N et al. “Failure of bone marrow to reconstitute lung epithelium.” Am J Respir Cell Mol Biol 2005; 33:328-334; Wagers A J, et al. “Little evidence for developmental plasticity of adult hematopoietic stem cells.” Science 2002; 297:2256-2259; Chang J C, et al. “Evidence that bone marrow cells do not contribute to the alveolar epithelium.” Am J Respir Cell Mol Biol 2005; 33:335-342). In addition, other reports have described that hematopoietic stem cells derived from bone marrow administered via an intranasal route results in alveolar macrophages, and that this population does not transdifferentiate into respiratory epithelial cells (Fritzell J A et al., Am J Respir Cell Mol Biol 2009 “Fate and Effects of Adult Bone Marrow Cells in Lungs of Normoxic and Hyperoxic Newborn Mice.” Vol.40, p. 575-587).

In contrast to these reports, the present inventor has discovered, in part, that umbilical cord blood derived hematopoietic stem cell population administered to the lungs result in repair and regeneration of lung tissue, including pulmonary alveoli, plulmonary vasculature, pulmonary endothelium, and pulmonary epithelial tissue. Accordingly, described herein are methods for lung repair, reconstitution, and regeneration involving intrapulmonary or systemic administration of umbilical cord blood-derived hematopoietic stem cells alone, such as CD34⁺ hematopoietic stem cells, in the absence of mesenchymal stem cells. Hematopoietic Stem Cells

In some aspects described herein, pluripotent hematopoietic stem and progenitor cells are isolated from a hematopoietic source, such as umbilical cord blood, circulating peripheral blood, bone marrow, fetal liver, or yolk sac of a mammal, for administration to a subject in need thereof.

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Hematopoietic stem cells (HSCs), as the term is used herein, refers to a subset of multipotent stem cells that give rise to all the blood or immune cell types, including myeloid (monocytes and macrophages, neutrophils, basophils, eosinophils, erythrocytes, megakaryocytes/platelets, dendritic cells), and lymphoid lineages (T-cells, B-cells, NKT-cells, NK-cells).

Hematopoietic tissues contain cells with long-term and short-term regeneration capacities, and committed multipotent, oligopotent, and unipotent progenitors. HSCs can be isolated or obtained from a variety of tissue sources, such as the bone marrow of adults, which includes femurs, hip, ribs, sternum, and other bones, as well as umbilical cord blood and placenta, and mobilized peripheral blood. HSCs can be obtained directly by removal from, for example, the hip using a needle and syringe, or from the blood following pre-treatment with cytokines, such as G-CSF (granulocyte colony-stimulating factors), that induce cells to be released from the bone marrow compartment.

Accordingly, “hematopoietic stem cells,” as used in the methods described herein, encompasses all pluripotent cells capable of differentiating into several cell types of the hematopoietic system, including, but not limited to, granulocytes, monocytes, erythrocytes, megakaryocytes, B-cells and T-cells. “Hematopoietic progenitor cells,” as the term is used herein, refer to the subset of hematopoietic stem cells that are committed to the hematopoietic cell lineage and generally do not self-renew, and can be identified, for example by cell surface markers such as Lin⁻KLS⁺Flk2⁻CD34⁺. The term “hematopoietic progenitor cells” encompasses short term hematopoietic stem cells (ST-HSCs), multi-potent progenitor cells (MPPs), common myeloid progenitor cells (CMPs), granulocyte-monocyte progenitor cells (GMPs), and megakaryocyte-erythrocyte progenitor cells (MEPs). Hematopoietic stem cells also include those long term hematopoietic stem cells that can be identified with the following stem cell marker profile: Lin⁻KLS⁺Flk2⁻CD34⁺. These subsets can also be identified on the basis of additional cell-surface marker phenotypes, such as, long-term hematopoietic stem cells (HSC): CD150⁺CD48⁻CD244⁻; MPPs: CD150⁻CD48⁻CD244⁺; lineage-restricted progenitor cells (LRPs): CD150⁻CD48⁺CD244⁺; common myeloid progenitor cells (CMP): lin⁻SCA-1⁻c-kit⁺CD34⁺CD16/32^{mid}; granulocyte-macrophage progenitor (GMP): lin⁻SCA-1⁻c-kit⁺CD34⁺CD16/32^{hi}; and megakaryocyte-erythroid progenitor (MEP): lin⁻SCA-1⁻c-kit⁺CD34⁺CD16/32^{low}. In some embodiments, hematopoietic stem cells used in the methods described herein are selected for, enriched for, or isolated using one or more of these additional cell surface markers.

The presence of hematopoietic progenitor cells can be determined functionally as colony forming unit cells (CFU-Cs) in complete methylcellulose assays, or phenotypically through the detection of cell surface markers using assays known to those of skill in the art. As used herein, the term “hematopoietic stem cell (HSC)” refers to a cell with multi-lineage hematopoietic differentiation potential and sustained self-renewal activity. “Self renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Hematopoietic stem cells have the ability to regenerate long term multi-lineage hematopoiesis (e.g., “long-term engraftment”) in individuals receiving a bone marrow or umbilical cord blood transplant.

The hematopoietic stem cells used for the various aspects described herein can be derived or isolated from any one or more of the following sources: fetal tissues, umbilical cord blood and/or placenta, bone marrow, peripheral blood, mobilized peripheral blood, a stem cell line, or can be derived ex vivo from other cells, such as embryonic stem cells, induced pluripotent stem cells (iPS cells) or adult pluripotent cells.

In some embodiments, the cells from the biological sources described herein can be expanded ex vivo using any method acceptable to those skilled in the art prior to use in the methods described herein. Further, the cells can be sorted, fractionated, treated to remove malignant cells, or otherwise manipulated to treat the patient using any procedure acceptable to those skilled in the art of preparing cells for transplantation.

As used herein, the term “population of hematopoietic cells” encompasses a heterogeneous or homogeneous population of hematopoietic stem cells and/or hematopoietic progenitor cells. In addition, differentiated hematopoietic cells, such as lymphocytes, can be present in a population of hematopoietic cells; that is, in some embodiments, hematopoietic stem and/or progenitor cells are not isolated from e.g., umbilical cord blood or bone marrow. A population of hematopoietic cells comprising at least two different cell types is referred to herein as a “heterogeneous population”. It is also contemplated herein that hematopoietic stem cells or hematopoietic progenitor cells are isolated and expanded ex vivo prior to transplantation. A population of hematopoietic cells comprising only one cell type (e.g., hematopoietic stem cells) is referred to herein as a “homogeneous population of cells”. The cell populations useful according to the methods described herein do not contain mesenchymal stem cells. Isolation of HSCs

Hematopoietic stem cells for use in the methods and uses described herein can be enriched for or isolated from a biological sample, preferably umbilical cord blood, using any method known to one of skill in the art.

The term “biological sample” as used herein refers to a cell or population of cells or a quantity of tissue or fluid from a subject comprising one or more hematopoietic stem cells. Most often, the biological sample has been removed from a subject, but the term “biological sample” can also refer to cells or tissue analyzed in vivo, i.e., without removal from the subject. Biological samples include, but are not limited to, umbilical cord blood, whole blood, bone marrow, tissue sample or biopsies, scrapes (e.g. buccal scrapes), plasma, serum, urine, saliva, cell culture, or cerebrospinal fluid. A biological sample or tissue sample can refer to any sample of tissue or fluid isolated from a subject from which hematopoietic stem cells can be obtained, including but not limited to, for example, umbilical cord blood, peripheral blood, bone marrow, thymus, lymph nodes, splenic tissue, liver tissue, plasma, sputum, serum, lung lavage fluid, tumor biopsy, urine, stool, spinal fluid, pleural fluid, nipple aspirates, lymph fluid, the external sections of the skin, respiratory, intestinal, and genitourinary tracts, tears, saliva, milk, cells (including but not limited to hematopoietic cells), tumors, organs, and also samples obtained from in vitro cell cultures.

In some embodiments of the aspects described herein, a biological sample comprising hematopoietic stem cells refers to a sample isolated from a subject, such as umbilical cord blood, peripheral blood, thymus, or bone marrow, which is then further processed, for example, by cell sorting (e.g., magnetic sorting or FACS), to obtain a population of hematopoietic stem cells. In other embodiments of the aspects described herein, a biological sample comprising hematopoietic stem cells refers to an in vitro or ex vivo culture of expanded hematopoietic stem cells. In some embodiments, a biological sample comprises an induced stem cell population, such as an induced pluripotent stem (iPS) cell population, as understood by one of skill in the art. In addition, fine needle aspirate samples are used. Samples can be frozen samples, such as frozen or cryopreserved umbilical cord blood samples. The sample can be obtained by removing a sample of cells from a subject, but can also be accomplished by using previously isolated cells (e.g., isolated from another subject), or by performing the methods described herein in vivo.

In some embodiments of the aspects described herein, the hematopoietic stem cells are isolated prior to their administration to a subject in need thereof. Such isolation can result in a substantially pure or enriched cell population for administration to the subject.

The terms “isolate” and “methods of isolation,” as used herein, refer to any process whereby a cell or population of cells, such as a population of hematopoietic stem cells, is removed from a subject or sample in which it was originally found, or a descendant of such a cell or cells. The term “isolated population,” as used herein, refers to a population of cells that has been removed and separated from a biological sample, or a mixed or heterogeneous population of cells found in such a sample. Such a mixed population includes, for example, a population of hematopoietic stem cells obtained from umbilical cord blood, or a cell suspension of a tissue sample. In some embodiments, an isolated population is a substantially pure population of cells as compared to the heterogeneous population from which the cells were isolated or enriched from. In some embodiments of this aspect and all aspects described herein, the isolated population is an isolated population of hematopoietic stem cells. In other embodiments of this aspect and all aspects described herein, the isolated population comprises a substantially pure population of hematopoietic stem cells as compared to a heterogeneous population of cells comprising various other cells types from which the hematopoietic stem cells were derived. In some embodiments, an isolated cell or cell population, such as a population of hematopoietic stem cells, is further cultured in vitro or ex vivo, e.g., in the presence of growth factors or cytokines, to further expand the number of cells in the isolated cell population or substantially pure cell population. Such culture can be performed using any method known to one of skill in the art, for example, as described in the Examples section. In some embodiments, the isolated or substantially pure hematopoietic stem cells populations obtained by the methods disclosed herein are later administered to a second subject, or re-introduced into the subject from which the cell population was originally isolated (e.g., allogenic transplantation vs. autologous administration).

The term “substantially pure,” with respect to a particular cell population, refers to a population of cells that is at least about 75%, at least about 80%, at least about 85%, at least about 90%, at least about 95%, at least about 98%, or at least about 99% pure, with respect to the cells making up a total cell population. In other words, the terms “substantially pure” or “essentially purified,” with regard to a population of hematopoietic stem cells isolated for use in the methods disclosed herein, refers to a population of hematopoietic stem cells that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 4%, fewer than about 3%, fewer than about 2%, fewer than about 1%, or less than 1%, of cells that are not hematopoietic stem cells, as defined by the terms herein. Some embodiments of these aspects further encompass methods to expand a population of substantially pure or enriched hematopoietic stem cells, wherein the expanded population of hematopoietic stem cells is also a substantially pure or enriched population of hematopoietic stem cells.

The terms “enriching” or “enriched” are used interchangeably herein and mean that the yield (fraction) of cells of one type, such as hematopoietic stem cells for use in the methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. A population of hematopoietic stem cells obtained for use in the methods described herein is most preferably at least 60% enriched for hematopoietic stem cells.

In some embodiments of the aspects described herein, markers specific for hematopoietic stem cells are used to isolate or enrich for these cells. A “marker,” as used herein, describes the characteristics and/or phenotype of a cell. Markers can be used for selection of cells comprising characteristics of interest. Markers will vary with specific cells. Markers are characteristics, whether morphological, functional or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, appearance (e.g., smooth, translucent), and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

Accordingly, as used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. A cell-surface marker of particular relevance to the methods described herein is CD34. The useful hematopoietic stem cells according to the present invention preferably express DC34 or in other words, they are CD34 positive.

A cell can be designated “positive” or “negative” for any cell-surface marker, and both such designations are useful for the practice of the methods described herein. A cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the methods described herein, the cell must express it on its surface. Similarly, a cell is considered “negative” for a cell-surface marker if it doe not express the marker on its cell-surface in amounts sufficient to be detected using methods known to those of skill in the art, such as contacting a cell with an antibody that binds specifically to that marker and subsequently performing flow cytometric analysis of such a contacted cell to determine whether the antibody is bound the cell. In some embodiments, where agents specific for cell-surface lineage markers used, the agents can all comprise the same label or tag, such as fluorescent tag, and thus all cells positive for that label or tag can be excluded or removed, to leave uncontacted hematopoietic stem or progenitor cells for use in the methods described herein.

Accordingly, as defined herein, an “agent specific for a cell-surface marker” refers to an agent that can selectively react with or bind to that cell-surface marker, but has little or no detectable reactivity to another cell-surface marker or antigen. For example, an agent specific for CD34 will not identify or bind to CD35. Thus, agents specific for cell-surface markers recognize unique structural features of the markers. In some embodiments, an agent specific for a cell-surface marker binds to the cell-surface marker, but does not cause initiation of downstream signaling events mediated by that cell-surface marker, for example, a non-activating antibody. Agents specific for cell-surface molecules include, but are not limited to, antibodies or antigen-binding fragments thereof, natural or recombinant ligands, small molecules; nucleic acid sequence and nucleic acid analogues; intrabodies; aptamers; and other proteins or peptides.

In some embodiments of this aspect and all aspects described herein, the preferred agents specific for cell-surface markers used for isolating hematopoietic stem cells are antibody agents that specifically bind the cell-surface markers, and can include polyclonal and monoclonal antibodies, and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Accordingly, as used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)]. Such antibodies or antigen-binding fragments are available commercially from vendors such as R&D Systems, BD Biosciences, e-Biosciences and Miltenyi, or can be raised against these cell-surface markers by methods known to those skilled in the art.

In some embodiments of the aspects described herein, an agent specific for a cell-surface molecule, such as an antibody or antigen-binding fragment, is labeled with a tag to facilitate the isolation of the hematopoietic stem cells. The terms “label” or “tag”, as used herein, refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and enrich endothelial cell progenitor cells.

The terms “labeled antibody” or “tagged antibody”, as used herein, includes antibodies that are labeled by detectable means and include, but are not limited to, antibodies that are fluorescently, enzymatically, radioactively, and chemiluminescently labeled. Antibodies can also be labeled with a detectable tag, such as c-Myc, HA, VSV-G, HSV, FLAG, V5, or HIS, which can be detected using an antibody specific to the tag, for example, an anti-c-Myc antibody. Various methods of labeling polypeptides and glycoproteins are known in the art and may be used. Non-limiting examples of fluorescent labels or tags for labeling the antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, nanoparticles, or quantum dots.

In some embodiments of the aspects described herein, a variety of methods to isolate a substantially pure or enriched population of hematopoietic stem cells are available to a skilled artisan, including immunoselection techniques, such as high-throughput cell sorting using flow cytometric methods, affinity methods with antibodies labeled to magnetic beads, biodegradable beads, non-biodegradable beads, and antibodies panned to surfaces including dishes, and any combination of such methods.

In some embodiments of these aspects and all aspects described herein, isolation of and enrichment for populations of hematopoietic stem cells can be performed using bead based sorting mechanisms, such as magnetic beads. In such methods, the biological sample, such as umbilical cord blood, is contacted with magnetic beads coated with antibodies against one or more specific cell-surface antigens, such as CD34. This causes the cells in the sample expressing this antigen to attach to the magnetic beads. Afterwards the contacted cell solution is transferred to a strong magnetic field, such as a column or rack having a magnet. The cells attached to the beads (expressing the cell-surface marker) stay on the column or sample tube, while other cells (not expressing the cell-surface marker) flow through or remain in solution. Using this method, cells can be separated positively or negatively, or using a combination therein, with respect to the particular cell-surface markers.

In some embodiments of the aspects described herein, magnetic activated cell sorting (MACS) strategies are used for isolation and preselection of hematopoietic stem cells. In some such embodiments, the isolated hematopoietic stem cells are still coupled with the microbead-bound antibodies when administered to a subject in need. In some embodiments, hematopoietic stem cells are isolated in the presence of human plasma or human serum albumin (HSA), such as 2% HSA.

In some preferred embodiments of the aspects described herein, HSCs are isolated or enriched using positive selection for the cell-surface marker CD34. As used herein, “CD34” refers to the protein that is a member of a family of single-pass transmembrane sialomucin proteins that show expression on early hematopoietic and vascular-associated tissue. CD34 also functions as an important adhesion molecule and is required for T cells to enter lymph nodes.

In other embodiments, one or more additional cell-surface markers are used for isolating and/or enriching for HSCs, using positive or negative selection methods, or a combination therein. Such additional cell-surface markers include, but are not limited to, CD133, lineage markers, KLS, Flk2, CD150, CD48, CD244, CD44, SCA-1, CD117 (c-kit), and CD16/32.

As defined herein, “positive selection” refers to techniques that result in the isolation or enrichment of cells expressing specific cell-surface markers, while “negative selection” refers techniques that result in the isolation or enrichment of cells not expressing specific cell-surface markers. In some embodiments, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select hematopoietic stem cells expressing one or more specific cell-surface markers. For example, in a negative selection protocol, a biological sample, such as a cell sample, is first contacted with labeled antibodies specific for cell-surface markers of interest, such as CD2, CD3, CD14, CD16, CD19, CD56, and CD235a and the sample is then contacted with beads that are specific for the labels of the antibodies, and the cells expressing any of the markers CD2, CD3, CD14, CD16, CD19, CD56, and CD235a are removed using immunomagnetic lineage depletion.

A number of different cell-surface markers have specific expression on specific differentiated cell lineages, and are not expressed by the hematopoietic stem cells isolated for the methods described herein. Accordingly, when agents specific for these lineage cell-markers are contacted with hematopoietic stem cells, the cells will be “negative.” Lineage cell-markers that are not expressed by the hematopoietic stem cells contemplated for use in the methods described herein include, but are not limited to, CD13 and CD33 (expressed on myeloid cells); CD71 (expressed on erythroid cells); CD19 and B220 (expressed on B cells), CD61 (expressed on human megakaryocytic cells); Mac-1 (CD11b/CD18) (expressed on monocytes); Gr-1 (expressed on granulocytes); Ter119 (expressed on erythroid cells); and Il7Ra, CD2, CD3, CD4, CD5, CD8 (expressed on T cells); CD14, CD56, and CD235a. In some embodiments of the aspects described herein, the lineage markers used can be dependent on the species from which the hematopoietic stem cells are being isolated, as determined by one of skill in the art. For example, when isolating human hematopoietic stem cells the combination of lineage markers to be excluded can comprise CD2, CD3, CD16, CD19, CD56, and CD235a. One can further enrich the cell population for the methods and uses described herein by removing cells that express the markers set forth in this paragraph.

Other embodiments of the aspects described herein use flow cytometric methods, alone or in combination with magnetic bead based methods, to isolate or enrich for hematopoetic stem cells. As defined herein, “flow cytometry” refers to a technique for counting and examining microscopic particles, such as cells and chromosomes, by suspending them in a stream of fluid and passing them through an electronic detection apparatus. Flow cytometry allows simultaneous multiparametric analysis of the physical and/or chemical parameters of up to thousands of particles per second, such as fluorescent parameters. Modern flow cytometric instruments usually have multiple lasers and fluorescence detectors. Increasing the number of lasers and detectors allows for labeling by multiple antibodies, and can more precisely identify a target population by their phenotypic markers. Certain flow cytometric instruments can take digital images of individual cells, allowing for the analysis of fluorescent signal location within or on the surface of cells.

A common variation of flow cytometric techniques is to physically sort particles based on their properties, so as to purify populations of interest, using “fluorescence-activated cell sorting” As defined herein, “fluorescence-activated cell sorting” or “flow cytometric based sorting” methods refer to flow cytometric methods for sorting a heterogeneous mixture of cells from a single biological sample into one or more containers, one cell at a time, based upon the specific light scattering and fluorescent characteristics of each cell and provides fast, objective and quantitative recording of fluorescent signals from individual cells as well as physical separation of cells of particular interest. Accordingly, in those embodiments when the agents specific for cell-surface markers are antibodies labeled with tags that can be detected by a flow cytometer, fluorescence-activated cell sorting (FACS) can be used in and with the methods described herein to isolate and enrich for populations of hematopoietic stem cells.

### Expansion of HSCs

In some embodiments of the aspects, the substantially pure or enriched for population of isolated hematopoietic stem cells are further expanded or increased in numbers prior to their use in the methods of treatment and uses described herein.

In some embodiments, hematopoietic stem cells isolated or enriched for using the methods and techniques described herein are expanded in culture, i.e., the cell numbers are increased, using methods known to one of skill in the art, prior to administration to a subject in need. In some embodiments, such expansion methods can comprise, for example, culturing the hematopoietic stem cells in serum-free medium supplemented with factors and/or under conditions that cause expansion of hematopoietic stem cells, such as stem cell factor, IL-3, and GM-CSF. In some embodiments of the methods described herein, hematopoietic stem cells are expanded in the presence of deaxmethasone. In some embodiments, hematopoietic stem cells can further be cultured with factors and/or under conditions aimed at inducing respiratory epithelial differentiation, such as using small airway growth medium, modified mouse tracheal epithelial cell medium, or serum-free medium supplemented with retinoic acid and/or keratinocyte growth factor. Some non-limiting expansion methods suitable for use with the methods described herein can be found in the Example section.

In other embodiments, hematopoietic stem cells isolated or enriched for use in the methods and techniques described herein are expanded using nanotechnological or nanoengineering methods, as reviewed in Lu J et al., “A Novel Technology for Hematopoietic Stem Cell Expansion using Combination of Nanofiber and Growth Factors.” Recent Pat Nanotechnol. 2010 Apr. 26.

For example, in some embodiments, nanoengineering of stem cell microenvironments can be performed. As used herein, secreted factors, stem cell - neighboring cell interactions, extracellular matrix (ECM) and mechanical properties collectively make up the “stem cell microenvironment.” Stem cell microenvironment nanoengineering can comprise the use of micro/nanopatterned surfaces, nanoparticles to control release growth factors and biochemicals, nanofibers to mimic extracellular matrix (ECM), nanoliter-scale synthesis of arrayed biomaterials, self-assembly peptide system to mimic signal clusters of stem cells, nanowires, laser fabricated nanogrooves, and nanophase thin films to expand hematopoietic stem cells.

In other embodiments, nanoengineering can be used for hematopoietic stem cell transfection and genetic manipulation in hematopoietic stem cells, such as nanoparticles for in vivo gene delivery, nanoneedles for gene delivery to hematopoietic stem cells, self-assembly peptide system for hematopoietic stem cell transfection, nanowires for gene delivery to hematopoietic stem cells, and micro/nanofluidic devices for hematopoietic stem cell electroporation

In other embodiments, hematopoietic stem cells isolated or enriched for use in the methods and techniques can be expanded using bioreactors.

The terms “increased,” “increase,” “enhance,” or “expand” are all used herein to generally mean an increase in the number of hematopoietic stem cells by a statically significant amount; for the avoidance of any doubt, the terms “increased,” “increase,” “expand,” “expanded,” or “enhance” mean an increase, as compared to a reference level, of at least about 10%, of at least about 15%, of at least about 20%, of at least about 25%, of at least about 30%, of at least about 35%, of at least about 40%, of at least about 45%, of at least about 50%, of at least about 55%, of at least about 6o%, of at least about 65%, of at least about 70%, of at least about 75%, of at least about 80%, of at least about 85%, of at least about 90%, of at least about 95%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, at least about a 10-fold increase, at least about a 25-fold increase, at least about a 50-fold increase, at least about a 100-fold increase, or any increase of 100-fold or greater, as compared to a control or reference level. A control sample or control level is used herein to describe a population of cells obtained from the same biological source that has, for example, not been expanded using the methods described herein.

### Umbilical Cord Blood

In preferred embodiments of the aspects described herein, human umbilical cord blood cells (UCB cells) or cord blood cells are useful as a source of hematopoietic stem and progenitor cells for administration to a subject in need. Human UBC cells are recognized as a rich source of hematopoietic and mesenchymal progenitor cells (Broxmeyer et al., 1992 Proc. Natl. Acad. Sci. USA 89:4109-4113). Cord blood cells can be used as a source of transplantable hematopoietic stem and progenitor cells for the various aspects described herein. Accordingly, in some embodiments, a biological sample from which a population of hematopoietic stem cells can be isolated or enriched from is an umbilical cord blood sample. Cord blood cells have been used as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503).

One distinct advantage of human umbilical cord blood cells over other sources of hematopoietic stem cells, such as bone marrow, for use in the methods of treatment described herein, is the immature immunity of these cells is very similar to fetal cells, which significantly reduces the risk for rejection by the host (Taylor & Bryson, 1985 J. Immunol. 134:1493-1497). Human umbilical cord blood contains, in addition to hematopoietic stem and progenitor cells, mesenchymal and endothelial cell precursors that can also be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). To obtain the best results with the methods described herein, the mesechymal stem cells are substantially removed from the hematopoietic cell population. Accordingly, in some embodiments of the methods and uses described herein, mesechymal stem cells are substantially removed from the hematopoietic cell population.

Moreover, the total content of hematopoietic progenitor cells in umbilical cord blood equals or exceeds that found in bone marrow samples. In addition, a population of highly proliferative hematopoietic cells are eightfold higher in human umbilical cord blood cells than in bone marrow, and express hematopoietic markers such as CD34, CD14, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

Additional advantages of human umbilical cord blood cells as a source of hematopoietic stem cells include, but are not limited to, autocrine production of hematopoietic growth factors, longer telomeres than are found in HSCs isolated from bone marrow or peripheral blood, lower infection rates, ethical acceptance, and better engraftment capabiltites.

In some embodiments of the aspects described herein, the umbilical cord is punctured with a needle, and the umbilical cord blood is collected in a conventional blood collection bag. In some embodiments, the umbilical cord blood is collected while the placenta is still in the uterus, while in other embodiments the umbilical cord blood is collected after the delivery of the placenta. In some embodiments of the aspects described herein, the last batch of umbilical cord blood, which is the batch collected before the cord is totally flushed, is used as the biological source of from which HSCs, such as CD34⁺ HSCs, are isolated or enriched from. Storage of Umbilical Cord Blood and/or Umbilical Cord Blood Cells

In some embodiments, the umbilical cord blood cells for use in the methods and uses described herein are stored prior to use. In some embodiments, whole umbilical cord blood is stored. In other embodiments, hematopoietic stem cells, such as CD34⁺ HSCs, are first isolated and/or expanded prior to storage.

In some embodiments, the umbilical cord blood cells or isolated hematopoietic stem cells are frozen prior to their use in the aspects described herein. Freezing the samples can be performed in the presence of one or more different cryoprotectants for minimizing cell damage during the freeze—thaw process. For example, dimethyl sulfoxide (DMSO), trehalose, or sucrose can be used.

### Administration and Uses of HSCs in Regenerative Medicine

Certain aspects of the invention described herein are based, in part, on the discovery by the inventor that administration of a population of hematopoietic stem cells isolated from umbilical cord blood, such as a CD34+ hematopoietic stem cell population, directly to the lung and airways results in long-term engraftment of the administered cells and differentiation into respiratory epithelium and consequent lung growth and alveolar regeneration and repair. The engraftment of the CD34+ hematopoietic stem cell population was also found to not require the presence or co-administration of additional cell types, such as mesenchymal cells. Further, the inventor found that administration of such hematopoietic stem cells isolated from umbilical cord blood via a systemic route, e.g., an intraperitoneal route, enhances pulmonary vascular regeneration and alveolar development. The inventor in addition discovered that arming of such hematopoietic stem cells from umbilical cord blood with a bispecific antibody, which targets or is specific for both the hematopoietic stem cells and a target tissue, serves as a therapeutic agent that can enhance homing of the hematopoietic stem cells to the target tissue upon systemic administration, for example, intraperitoneal administration and intrapulmonary administration.

Accordingly, provided herein are methods for the treatment and prevention of a respiratory disease or disorder in a subject in need thereof. Some of these methods involve administering to a subject a therapeutically effective amount of hematopoietic stem cells using intrapulmonary administration, such as an intransal or intratracheal route. In some aspects of these methods, a therapeutically effective amount of hematopoietic stem cells is administered using a systemic, such as an intraperitoneal or intravenous route. In other aspects of these methods, a therapeutically effective amount of hematopoietic stem cells is administered using both intrapulmonary and intraperitoneal administration. These methods are particularly aimed at therapeutic and prophylactic treatments of human subjects having or at risk for a respiratory disease or disorder. The isolated or enriched hematopoietic stem cells described herein can be administered to a subject having any respiratory disease or disorder by any appropriate route which results in an effective treatment in the subject. In some embodiments of the aspects described herein, a subject having a respiratory disorder is first selected prior to administration of the cells.

The terms “subject” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

Accordingly, for the various embodiments of the methods described herein, a subject is a recipient subject, i.e., a subject to whom the hematopoietic stem cells are being administered, or a donor subject, i.e., a subject from whom a biological sample comprising hematopoietic stem cells are being obtained. A recipient or donor subject can be of any age. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In some embodiments of the aspects described herein, a newborn subject is defined as a subject less than 24 hours of age. A “premature infant subject” is any subject born before 37 weeks, before 36 weeks, before 35 weeks, before 34 weeks, before 33 weeks, before 32 weeks, before 31 weeks, before 30 weeks, before 29 weeks, before 28 weeks, before 27 weeks, before 26 weeks, before 25 weeks, before 24 weeks, before 23 weeks, before 22 weeks, before 21 weeks, or before 20 weeks of gestation.

In some embodiments of the aspects described herein, the hematopoietic stem cell population being administered according to the methods described herein, comprises allogeneic hematopoietic stem cells obtained from one or more donors. As used herein, “allogeneic” refers to hematopoietic stem cell or biological samples comprising hematopoietic stem cell obtained from one or more different donors of the same species, where the genes at one or more loci are not identical. For example, a hematopoietic stem cell population being administered to a subject can be obtained from umbilical cord blood obtained from one more unrelated donor subjects, or from one or more non-identical siblings. In some embodiments, syngeneic hematopoietic stem cell populations can be used, such as those obtained from genetically identical animals, or from identical twins. In other embodiments of this aspect, the hematopoietic stem cells are autologous hematopoietic stem cells. As used herein, “autologous” refers to hematopoietic stem cells or biological samples comprising hematopoietic stem cells obtained or isolated from a subject and being administered to the same subject, i.e., the donor and recipient are the same.

The methods described herein can be used to treat, ameliorate, prevent or slow the progression of a number of respiratory diseases or their symptoms, such as those resulting in pathological damage to lung or airway architecture and/or alveolar damage. The terms “respiratory disorder,” “respiratory disease,” “pulmonary disease,” and “pulmonary disorder,” are used interchangeably herein and refer to any condition and/or disorder relating to respiration and/or the respiratory system, including the lungs, pleural cavity, bronchial tubes, trachea, upper respiratory tract, airways, or other components or structures of the respiratory system. Such respiratory diseases include, but are not limited to, bronchopulmonary dysplasia (BPD), chronic obstructive pulmonary disease (COPD) condition, cystic fibrosis, bronchiectasis, cor pulmonale, pneumonia, lung abcess, acute bronchitis, chronic bronchitis, emphysema, pneumonitis, e.g., hypersensitivity pneumonitis or pneumonitis associated with radiation exposure, alveolar lung diseases and interstitial lung diseases, environmental lung disease (e.g., associated with asbestos, fumes or gas exposure), aspiration pneumonia, pulmonary hemorrhage syndromes, amyloidosis, connective tissue diseases, systemic sclerosis, ankylosing spondylitis, pulmonary actinomycosis, pulmonary alveolar proteinosis, pulmonary anthrax, pulmonary edema, pulmonary embolus, pulmonary inflammation, pulmonary histiocytosis X, pulmonary hypertension, surfactant deficiencies, pulmonary hypoplasia, pulmonary neoplasia, pulmonary nocardiosis, pulmonary tuberculosis, pulmonary veno-occlusive disease, rheumatoid lung disease, sarcoidosis, post-pneumonectomy, Wegener's granulomatosis, allergic granulomatosis, granulomatous vasculitides, eosinophilia, asthma and airway hyperreactivity (AHR), e.g., mild intermittent asthma, mild persistent asthma, moderate persistent asthma, severe persistent asthma, acute asthma, chronic asthma, atopic asthma, allergic asthma or idiosyncratic asthma, cystic fibrosis and associated conditions, e.g., allergic bronchopulmonary aspergillosis, chronic sinusitis, pancreatic insufficiency, lung or vascular inflammation, bacterial or viral infection, e.g., Haemophilus influenzae, S. aureus, Pseudomonas aeruginosa or respiratory syncytial virus (RSV) infection or an acute or chronic adult or pediatric respiratory distress syndrome (RDS) such as grade I, II, III or IV RDS or an RDS associated with, e.g., sepsis, pneumonia, reperfusion, atelectasis or chest trauma.

Chronic obstructive pulmonary diseases (COPDs) include those conditions where airflow obstruction is located at upper airways, intermediate-sized airways, bronchioles or parenchyma, which can be manifested as, or associated with, tracheal stenosis, tracheal right ventricular hypertrophy pulmonary hypertension, polychondritis, bronchiectasis, bronchiolitis, e.g., idiopathic bronchiolitis, ciliary dyskinesia, asthma, emphysema, connective tissue disease, bronchiolitis of chronic bronchitis or lung transplantation.

The methods described herein can also be used to treat or ameliorate acute or chronic asthma or their symptoms or complications, including airway epithelium injury, airway smooth muscle spasm or airway hyperresponsiveness, airway mucosa edema, increased mucus secretion, excessive, T cell activation, or desquamation, atelectasis, cor pulmonale, pneumothorax, subcutaneous emphysema, dyspnea, coughing, wheezing, shortness of breath, tachypnea, fatigue, decreased forced expiratory volume in the 1st second (FEV₁), arterial hypoxemia, respiratory acidosis, inflammation including unwanted elevated levels of mediators such as IL-4, IL-5, IgE, histamine, substance P, neurokinin A, calcitonin gene-related peptide or arachidonic acid metabolites such as thromboxane or leukotrienes (LTD₄ or LTC₄), and cellular airway wall infiltration, e.g., by eosinophils, lymphocytes, macrophages or granulocytes.

Any of these and other respiratory or pulmonary conditions or symptoms are described elsewhere, e.g., The Merck Manual, 17.sup.th edition, M. H. Beers and R. Berkow editors, 1999, Merck Research Laboratories, Whitehouse Station, N.J., ISBN 0911910-10-7, or in other references cited herein. In some of these conditions, where inflammation plays a role in the pathology of the condition, the methods described herein can ameliorate or slow the progression of the condition by reducing damage from inflammation, such as damage to the lung epithelium. In other cases, the methods described herein act to limit pathogen replication or pathogen-associated lung tissue damage.

As used herein, the terms “administering,” “introducing” and “transplanting” are used interchangeably in the context of the placement of cells, e.g. hematopoietic stem cells, of the invention into a subject, by a method or route which results in at least partial localization of the introduced cells at a desired site, such as a site of injury or repair, such that a desired effect(s) is produced. The cells e.g. hematopoietic stem cells, or their differentiated progeny (e.g. respiratory epithelium-like cells) can be implanted directly to the respiratory airways, or alternatively be administered by any appropriate route which results in delivery to a desired location in the subject where at least a portion of the implanted cells or components of the cells remain viable. The period of viability of the cells after administration to a subject can be as short as a few hours, eg., twenty-four hours, to a few days, to as long as several years, i.e., long-term engraftment. For example, in some embodiments of the aspects described herein, an effective amount of an isolated or enriched population of hematopoietic stem cells is administered directly to the lungs of an infant suffering from bronchopulmonary dysplasia by intratracheal administration. In other embodiments, isolated or enriched hematopoietic stem cells can be administered via an indirect systemic route of administration, such as an intraperitoneal or intravenous route.

When provided prophylactically, isolated or enriched hematopoietic stem cells can be administered to a subject in advance of any symptom of a respiratory disorder, e.g., asthma attack or to a premature infant. Accordingly, the prophylactic administration of an isolated or enriched for hematopoietic stem cell population serves to prevent a respiratory disorder, as disclosed herein.

When provided therapeutically, isolated or enriched hematopoietic stem cells are provided at (or after) the onset of a symptom or indication of a respiratory disorder, e.g., upon the onset of COPD.

Accordingly, as used herein, the terms “treat,” “treatment,” “treating,” “prevention” or “amelioration” refer to both therapeutic treatment and prophylactic or preventative measures, wherein the object is to prevent, delay the onset, reverse, alleviate, ameliorate, inhibit, or slow down the progression or severity of a condition associated with, a disease or disorder. The term “treating” includes reducing or alleviating at least one adverse effect or symptom of a condition, disease or disorder associated with an inflammatory disease, such as, but not limited to, asthma. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable.

The term “treatment” of a disease also includes providing relief from the symptoms or side-effects of the disease (including palliative treatment). For example, any reduction in inflammation, bronchospasm, bronchoconstriction, shortness of breath, wheezing, lower extremity edema, ascites, productive cough, hemoptysis, or cyanosis in a subject suffering from a respiratory disorder, such as asthma, no matter how slight, would be considered an alleviated symptom. In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 90%, upon administration of a population of isolated or enriched for hematopoietic stem cells, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated. “Treatment” can also mean prolonging survival as compared to expected survival if not receiving treatment. Thus, one of skill in the art realizes that a treatment may improve the disease condition, but may not be a complete cure for the disease.

The term “effective amount” as used herein refers to the amount of a population of isolated or enriched for hematopoietic stem cells needed to alleviate at least one or more symptom of the respiratory disease or disorder, and relates to a sufficient amount of pharmacological composition to provide the desired effect, i.e., treat a subject having bronchopulmonary dysplasia. The term “therapeutically effective amount” therefore refers to an amount isolated or enriched for hematopoietic stem cells using the methods as disclosed herein that is sufficient to effect a particular effect when administered to a typical subject, such as one who has or is at risk for bronchopulmonary dysplasia. An effective amount as used herein would also include an amount sufficient to prevent or delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using routine experimentation.

In some embodiments of the invention, the subject is first diagnosed as having a disease or disorder affecting the lung tissue prior to administering the cells according to the methods described herein. In some embodiments, the subject is first diagnosed as being at risk of developing lung disease or disorder prior to administering the cells. For example, a premature infant may be at a significant risk of developing a lung disease or disorder.

For use in the various aspects described herein, an effective amount of hematopoietic stem cells, or an enriched fraction thereof, comprises at least 10² hematopoietic stem cells, at least 5×10² hematopoietic stem cells, at least 10³ hematopoietic stem cells, at least 5×10³ hematopoietic stem cells, at least 10⁴ hematopoietic stem cells, at least 5×10⁴ hematopoietic stem cells, at least 10⁵ hematopoietic stem cells, at least 2×10⁵ hematopoietic stem cells, at least 3×10⁵ hematopoietic stem cells, at least 4×10⁵ hematopoietic stem cells, at least 5×10⁵ hematopoietic stem cells, at least 6×10⁵ hematopoietic stem cells, at least 7×10⁵ hematopoietic stem cells, at least 8×10⁵ hematopoietic stem cells, at least 9×10⁵ hematopoietic stem cells, at least 1×10⁶ hematopoietic stem cells, at least 2×10⁶ hematopoietic stem cells, at least 3×10⁶ hematopoietic stem cells, at least 4×10⁶ hematopoietic stem cells, at least 5×10⁶ hematopoietic stem cells, at least 6×10⁶ hematopoietic stem cells, at least 7×10⁶ hematopoietic stem cells, at least 8×10⁶ hematopoietic stem cells, at least 9×10⁶ hematopoietic stem cells, or multiples thereof. The hematopoietic stem cells can be isolated or enriched for from one or more donors, or can be obtained from an autologous source. In some embodiments of the aspects described herein, the hematopoietic stem cells are an expanded population of cells.

Effective amount, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage may vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose may be formulated in animal models to achieve a circulating plasma concentration range that includes the IC50, which achieves a half-maximal inhibition of symptoms as determined in cell culture, or in an appropriate animal model. The effects of any particular dosage can be monitored by a suitable bioassay. The dosage may be determined by a physician and adjusted, as necessary, to suit observed effects of the treatment.

Exemplary modes of administration for use in the methods described herein include, but are not limited to, injection, intrapulmonary (including intranasal and intratracheal) infusion, inhalation (including intranasal), ingestion, and rectal administration. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “parenteral administration” and “administered parenterally” as used herein, refer to modes of administration other than enteral and topical administration, usually by injection, and includes, without limitation, intravenous, intraperitoneal, intramuscular, intraarterial, intrathecal, intraventricular, intracapsular, intraorbital, intracardiac, intradermal, transtracheal, subcutaneous, subcuticular, intraarticular, sub capsular, subarachnoid, intraspinal, intracerebro spinal, and intrasternal injection and infusion.

In preferred embodiments, an effective amount of cord blood-derived hematopoietic stem cells are administered to a subject by intrapulmonary administration or delivery. As defined herein, “intrapulmonary” administration or delivery refers to all routes of administration whereby a population of hematopoietic stem cells, such as CD34+ hematopoietic stem cells, is administered in a way that results in direct contact of these cells with the airways of a subject, including, but not limited to, transtracheal, intratracheal, and intranasal administration. In some such embodiments, the cells are injected into the nasal passages or trachea. In some embodiments, the cells are directly inhaled by a subject. In some embodiments, intrapulmonary delivery of cells includes administration methods whereby cells are administered, for example as a cell suspension, to an intubated subject via a tube placed in the trachea or “tracheal intubation.”

As used herein, “tracheal intubation” refers to the placement of a flexible tube, such as a plastic tube, into the trachea. The most common tracheal intubation, termed herein as “orotracheal intubation” is where, with the assistance of a laryngoscope, an endotracheal tube is passed through the mouth, larynx, and vocal cords, into the trachea. A bulb is then inflated near the distal tip of the tube to help secure it in place and protect the airway from blood, vomit, and secretions. In some embodiments, cells are administered to a subject having “nasotracheal intubation,” which is defined as a tracheal intubation where a tube is passed through the nose, larynx, vocal cords, and trachea.

In some embodiments, an effective amount of cord blood-derived hematopoietic stem cells are administered to a subject by systemic administration, such as intravenous administration.

The phrases “systemic administration,” “administered systemically”, “peripheral administration” and “administered peripherally” as used herein refer to the administration of population of hematopoietic stem cell other than directly into a target site, tissue, or organ, such as the lung, such that it enters, instead, the subject's circulatory system and, thus, is subject to metabolism and other like processes.

In some embodiments of the aspects described herein, one or more routes of administration are used in a subject to achieve distinct effects. For example, isolated or enriched population of hematopoietic stem cells are administered to a subject by both intratracheal and intraperitoneal administration routes for treating or repairing respiratory epithelium and for pulmonary vascular repair and regeneration respectively. In such embodiments, different effective amounts of the isolated or enriched hematopoietic stem cells can be used for each administration route.

In some embodiments of the aspects described herein, the methods further comprise administration of one or more therapeutic agents, such as a drug or a molecule, that can enhance or potentiate the effects mediated by the administration of the isolated or enriched hematopoietic stem cells, such as enhancing homing or engraftment of the hematopoietic stem cells, increasing repair of respiratory epithelia, or increasing growth and regeneration of pulmonary vasculature, i.e., vascular regeneration. The therapeutic agent may be a protein (such as an antibody or antigen-binding fragment), a peptide, a polynucleotide, an aptamer, a virus, a small molecule, a chemical compound, a cell, a drug, etc. As defined herein, “vascular regeneration” refers to the formation of new blood vessels or the replacement of damaged blood vessels (e.g., capillaries) after injuries or traumas, as described herein, including but not limited to, respiratory disease. “Angiogenesis” is a term that can be used interchangeably to described such phenomena.

Importantly, the inventor has discovered that arming of hematopoietic stem cells with a therapeutic agent, such as a bispecific antibody, enhances regeneration and repair of lung tissues and pulmonary vasculature. Bispecific antibody (BiAb) technology can combine an effector cell-specific antibody with an injury- or tissue-specific targeting antibody to create a biologic bridge for the purpose of directing cells with reparative or regenerative potential to injured or defective tissue. Described herein are bispecific antibodies for enhancing the homing of intranasally delivered stem cells to the alveolar epithelium. Following intranasal delivery and aspiration into the lungs, cells reside in a ‘free-floating’ state in the distal airspaces for variable amounts of time. By directed targeting of intranasally delivered stem cells to the alveolar epithelium, using bispecific antibodies as described herein, cell survival is enhanced and overall engraftment efficiency is improved.

“Arming” cells with a therapeutic agent can be performed, for example by incubating the cells with the therapeutic agent, such as a bi-specific antibody. Thus, cells are allowed to bind to the therapeutic agent, such as the antibody specific to the cells. Typically, the cells are thereafter washed to remove unbound therapeutic agents. Thus, as defined herein, “arming” of cells refers to any method wherein a cell for use in the methods described herein is contacted with a therapeutic agent that specifically binds to the cells. In preferred embodiments, the therapeutic agent is specific for the cell and for a molecule expressed on a site to which the cell is to home to.

In some embodiments, other homing agents can be used as therapeutic agents and can be similarly bound to the cells by a receptor-ligand interaction.

In some instances, cells can be genetically engineered to express molecules for homing or targeting, such as specific membrane bound receptor molecules or ligands. Such receptors and/or ligands may be engineered to have a cell membrane binding domain and an extracellular domain that will assist in homing of the cells. Methods for genetically engineering cells are well known to one skilled in the art.

Accordingly, in some embodiments, the methods further comprise administration of a antibody or antigen binding fragment for targeting a population of isolated or enriched hematopoietic stem cells being administered using any of the methods described herein to a desired respiratory target tissue in need of repair, for example, the lung alveoli. In some embodiments, the antibody is administered with a population of isolated or enriched hematopoietic stem cells being administered systemically, or using parenteral administration, such as intraperitoneally or by intrapulmonary administration. In some embodiments, the bispecific antibody is administered together with transtracheal, intratracheal, and intransal administration of the hematopoietic stem cells.

An antibody or antigen-binding fragment for use in such embodiments as a therapeutic agent can be any antibody or antigen-binding fragment specific for an antigen desired to be targeted to using the methods described herein, and can include polyclonal, monoclonal, and bispecific antibodies, and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. In some embodiments of the methods described herein, an antibody or antigen binding fragment is a bispecific antibody. A bispecific antibody refers to an antibody or fragment thereof that can bind to two distinct and unrelated antigens and is generated by combining parts of two separate antibodies that recognize two different antigenic groups. This may be achieved by crosslinking or recombinant techniques. Additionally, moieties may be added to the antibody or a portion thereof to increase half-life in vivo (e.g. , by lengthening the time to clearance from the blood stream. Such techniques include, for example, adding PEG moieties (also termed pegylation), and are well-known in the art. See U.S. Patent. Appl. Pub. 20030031671.

An exemplary bispecific antibody for use in arming the cells for the methods described herein is a bispecific antibody that is specific for an antigen on the hematopoietic stem cell (e.g., CD34) and specific for an antigen present on a target tissue, such as a VCAM-1 or e-cadherin (as an epithelial marker). The inventors have determined that VCAM-1 is expressed by both alveolar epithelium and pulmonary microvascular endothelium of newborn mice, both in normoxic and hyperoxic conditions. By using a bispecific antibody directed against VCAM-1 expressed in the underlying endothelium, adhesion of the stem cells to the alveolar lining, transmigration between epithelial cells, and endothelial engraftment are facilitated.

For example, to arm cells, hUCB-CD34+ cells can be incubated with the hCD34×mVCAM-1 BiAb (1000 ng per 10⁶ cells) and washed to remove unbound BiAb. BiAb binding or “arming” can be detected by staining with goat anti-rat IgG2a-FITC and analyzed, for example, by flow cytometry. The cells can be either freshly isolated, or cells that have undergone ex vivo expansion. Typically, one can expand the cells for about 1, 2, 3, 4, 5, 6, 7, 8, or even 10-days, or 1-10-days or 3-7-days. In some embodiments, the cells are armed after a 3-day expansion, when >95% of cells are still CD34+ when measured by, for example, FACS and/or immunohistochemistry.

In certain embodiments of these aspects, the therapeutic agent is a “pro-angiogenic factor,” which refers to factors that directly or indirectly promote new blood vessel formation. The pro-angiogenic factors include, but are not limited to epidermal growth factor (EGF), E-cadherin, VEGF, angiogenin, angiopoietin-1, fibroblast growth factors: acidic (aFGF) and basic (bFGF), fibrinogen, fibronectin, heparanase, hepatocyte growth factor (HGF), angiopoietin, hypoxia-inducible factor-1 (HIF-1), insulin-like growth factor-1 (IGF-1), IGF, BP-3, platelet-derived growth factor (PDGF), VEGF-A VEGF-C, pigment epithelium-derived factor (PEDF), vascular permeability factor (VPF), vitronection, leptin, trefoil peptides (TFFs), CYR61 (CCN1) and NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), pleitrophin, proliferin, follistatin, placental growth factor (PIGF), midkine, platelet-derived growth factor-BB (PDGF), and fractalkine, and inflammatory cytokines and chemokines that are inducers of angiogenesis and increased vascularity, eg. interleukin-3 (IL-3), interleukin-8 (IL-8), CCL2 (MCP-1), interleukin-8 (IL-8) and CCL5 (RANTES).

In some embodiments of the aspects described herein, the methods further comprise administration of one or more surfactants as therapeutic agents, or may be used in combination with one or more surfactant therapies. Surfactant, as used herein, refers to any surface active agent, including but not limited to wetting agents, surface tension depressants, detergents, dispersing agents, emulsifiers. Particularly preferred are those that from a monomolecular layer over pulmonary alveolar surfaces, including but not limited to lipoproteins, lecithins, and sphygomyelins. Exemplary surfactants include, but are not limited to surfactant protein A, surfactant protein B, surfactant protein C, surfactant protein D, and mixtures and combinations thereof. Commercially available surfactants include, but are not limited to, KL-4, Survanta, bLES, Infasurf, Curosurf, HL-10, Alveofact, Surfaxin, Venticute, Pumactant/ALEC, and Exosurf.

In some embodiments of the aspects described herein, administration of one or more other standard therapeutic agents can be combined with the administration of hematopoietic stem cells to treat the respiratory disorders or conditions, e.g., asthma, RDS or COPD, including the use of anticholinergic agents, β-2-adrenoreceptor agonists, such as formoterol or salmeterol, corticosteroids, antibiotics, anti-oxidation, antihypertension agents, nitric oxide, caffeine, dexamethasome, and IL-10 or other cytokines.

For example, the use of hematopoietic stem cells in the methods described herein to treat, ameliorate or slow the progression of a condition such as CF can be optionally combined with other suitable treatments or therapeutic agents. For CF, this includes, but is not limited to, oral or aerosol corticosteroid treatment, ibuprofen treatment, DNAse or IL-10 treatment, diet control, e.g., vitamin E supplementation, vaccination against pathogens, e.g., Haemophilus influenzae, chest physical therapy, e.g., chest drainage or percussion, or any combination therein.

The therapeutic methods described herein for the treatment of respiratory or pulmonary conditions using hematopoietic stem cells can be used in conjunction with other therapeutic agents and/or compositions that have been described in detail, see, e.g., Harrison's Principles of Internal Medicine, 15.sup.th edition, 2001, E. Braunwald, et al., editors, McGraw-Hill, New York, N.Y., ISBN 0-07-007272-8, especially chapters 252-265 at pages 1456-1526; Physicians Desk Reference 54.sup.th edition, 2000, pages 303-3251, ISBN 1-56363-330-2, Medical Economics Co., Inc., Montvale, N.J. Treatment of any of these respiratory and pulmonary conditions using a composition may be accomplished using the treatment regimens described herein. For chronic conditions, intermittent dosing can be used to reduce the frequency of treatment. Intermittent dosing protocols are as described herein.

### Manipulation of Homing and Engraftment of HSCs

Specific homing and engraftment of hematopoietic stem cells within the bone marrow, and potentially other organs, depend on a multistep series of events, involving the orchestrated expression and activation of a variety of chemokines, cytokines and adhesion molecules. Stromal derived factor-1 (SDF-1, also called CXCL12) along with its receptor, CXCR4, are among the most important peptides determining homing and engraftment of bone marrow-derived cells.

SDF-1 is a member of the CXC chemokine family and is highly conserved among species, including human and mouse. Human and murine SDF-1 are cross-reactive, enabling human CXCR4 to respond to murine SDF-1 signaling and vice versa. SDF-1 is produced by bone marrow stromal cells and also by epithelial cells in many other organs. SDF-1 is expressed in lung epithelium and its expression increases in injured lungs. The SDF-1 receptor, CXCR4, is expressed by a variety of cells, including immature hematopoietic cells. The SDF-1/CXCR4 axis is essential for bone marrow engraftment by human hematopoietic stem cells in NOD/SCID mice6. Accordingly, in some embodiments of the aspects described herein, the methods further comprise modulating the SDF-1/CXCR4 axis to enhance hematopoietic stem cell engraftment. In other embodiments, the methods further comprise modulating other adhesion molecules and their receptors, such as very late activation antigen-4 (VLA-4), VLA-5, leukocyte function antigen-1 (LFA-1) and their vascular ligands VCAM-1 and ICAM-1.

In some embodiments of the aspects described herein, the methods further comprise genetically engineering the isolated or enriched for population of hematopoietic stem cells, or their progenitor cells by modifying the genetic material of these cells or adding genetic material (e.g., DNA or RNA) of interest into these cells. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

For the clinical use of the methods described herein, isolated or enriched populations of hematopoietic stem cells described herein can be administered along with any pharmaceutically acceptable compound, material, or composition which results in an effective treatment in the subject. Thus, a pharmaceutical formulation for use in the methods described herein can contain an isolated or enriched population of hematopoietic stem cells in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or steric acid), or solvent encapsulating material, involved in maintaining the activity of, carrying, or transporting the isolated or enriched populations of hematopoietic stem cells from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) cellulose, and its derivatives, such as sodium carboxymethyl cellulose, methylcellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂⁻ C₁₂ alchols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

As used herein, in vivo (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating hematopoietic stem cells from umbilical cord blood obtained from a donor subject, and then administering the isolated hematopoietic stem cell sample to a recipient subject. As used herein, “in vitro” refers to those methods performed outside of a subject, such as an in vitro cell culture experiment. For example, isolated hematopoietic stem cells can be cultured in vitro to expand or increase the number of hematopoietic stem cells, or to direct differentiation of the hematopoietic stem cells to a specific lineage or cell type, e.g., respiratory epithelial cells, prior to being used or administered according to the methods described herein.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential.

The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

Embryonic stem cells and methods of their retrieval are well known in the art and are described, for example, in Trounson A 0 (Reprod Fertil Dev (2001) 13: 523), Roach M L (Methods Mol Biol (2002) 185: 1), and Smith A G (Annu Rev Cell Dev Biol (2001) 17:435). Adult stem cells are stem cells, which are derived from tissues of adults and are also well known in the art. Methods of isolating or enriching for adult stem cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. In some embodiments, adult stem cells can be of non-fetal origin. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a hematopoietic stem cell), which in turn can differentiate into other types of precursor cells further down the pathway (such as a thymocyte, or a T lymphocyte precursor), and then to an end-stage differentiated cell, which plays a characteristic role in a certain tissue type, and may or may not retain the capacity to proliferate further.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an hematopoietic stem cell) in a cellular differentiation process. Without wishing to be limited to theory, a pluripotent stem cell in the course of normal ontogeny can differentiate first to an endothelial cell that is capable of forming hematopoietic stem cells and other cell types. Further differentiation of a hematopoietic stem cell leads to the formation of the various blood or immune cell types, including myeloid (monocytes and macrophages, neutrophils, basophils, eosinophils, erythrocytes, megakaryocytes/platelets, dendritic cells), and lymphoid lineages (T-cells, B-cells, NKT cells, NK-cells).

As used herein, the term “somatic cell” refers to are any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro . In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” is used herein describes a cell with a common ancestry or cells with a common developmental fate. In the context of a cell that is of hematopoietic origin or is “hematopoietic linage” this means the cell was derived from a hematopoietic stem cell and can differentiate along lineage restricted pathways, such as one or more developmental lineage pathways which give rise to hematopoietic cells, which in turn can differentiate, for example, into T cells and B cells.

As used herein, the term “xenogeneic” refers to cells that are derived from different species.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “tissue” refers to a group or layer of specialized cells which together perform certain special functions. The term “tissue-specific” refers to a source of cells from a specific tissue.

The terms “decrease” , “reduced”, “reduction” , “decrease” or “inhibit” are all used herein generally to mean a decrease by a statistically significant amount. However, for avoidance of doubt, “”reduced“, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e. absent level as compared to a reference sample), or any decrease between 10-90% as compared to a reference level.

The terms “increased” ,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% increase or more or any increase between 10-90% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of additional elements that do not materially affect the basic and novel or functional characteristic(s) of that embodiment of the invention.

The term “consisting of refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

The role of umbilical cord blood-derived stem cell therapy in neonatal lung injury remains undetermined. As described herein, the inventors have investigated the capacity of human cord blood-derived CD34+ hematopoietic progenitor cells to regenerate injured alveolar epithelium in newborn mice. Double transgenic mice with doxycycline (Dox)-dependent lung-specific Fas-ligand (FasL) overexpression, treated with Dox between embryonal day 15 and postnatal day 3 (P3), served as model of neonatal lung injury. Single transgenic, non-Dox-responsive littermates were controls. CD34+ cells (1 to 5×10⁵) were administered at P5 by intranasal inoculation. Engraftment, respiratory epithelial differentiation, proliferation and cell fusion were studied at 8 weeks post-inoculation. Engrafted cells were readily detected in all recipients and showed a higher incidence of surfactant-immunoreactivity and proliferative activity in FasL-overexpressing animals compared with non-FasL-injured littermates. Cord blood-derived cells surrounding surfactant-immunoreactive type II-like cells frequently showed a transitional phenotype between type II and type I cells and/or type I cell-specific podoplanin immunoreactivity. Lack of nuclear colocalization of human and murine genomic material suggested absence of fusion. In conclusion, human cord blood-derived CD34+ cells are capable of long-term pulmonary engraftment, replication, clonal expansion, and reconstitution of injured respiratory epithelium by fusion-independent mechanisms. Cord blood-derived surfactant-positive epithelial cells act as progenitors of the distal respiratory unit, analogous to resident type II cells. Graft proliferation and alveolar epithelial differentiation are promoted by lung injury, as shown herein.

### Materials and Methods

Isolation of CD34⁺ cells from human cord blood. Human umbilical cord blood (hUCB) was obtained from uncomplicated full-term cesarean deliveries at Women and Infants Hospital according to IRB-approved protocol. Cord blood was collected in citrate phosphate dextrose (CPD) whole blood collection bags (Baxter Healthcare Corp., Deerfield, Ill.) and processed within 2 hours. Mononuclear cord blood cells (UCB-MNCs) were isolated by Ficoll-Hypaque density gradient centrifugation (Fisher BioReagents, Pittsburgh, Pa.). UCB-CD34⁺ cells were isolated from mononuclear cell suspensions by immunomagnetic cell sorting (MACS) according to the manufacturer's instructions (CD34 MicroBead Kit, Miltenyi Biotec, Bergisch Gladbach, Germany) (Lee, 2007;Zhao, 2008). CD34+ cell purity was determined by immunocytochemistry and flow cytometry analysis of the MACS product using a phycoerythrin-conjugated anti-human CD34 antibody (DAKO, Glostrup, Denmark). Cell viability was determined by Trypan blue exclusion.

The morphology of human cord blood-derived mononuclear and CD34+ cells were analyzed. Giemsa staining of cytocentrifuged cells obtained at successive steps of a CD34⁺ cell isolation procedure was performed. The appearance of mononuclear cell preparation obtained after density gradient centrifugation of human cord blood was investigated. The cells display marked pleomorphism, consistent with their derivation from myeloid, erythroid, and megakaryocyte lineages. The appearance of CD34⁺ cell isolates obtained after positive MACS sorting of CD34⁺ hematopoietic progenitor cells was also analyzed and the cells appeared significantly more homogeneous. Immunostaining of CD34+ cells was performed following MACS sorting using FITC-labeled anti-CD34 antibody. Anti-CD34 immunoreactivity was observed in >95% of MACS-sorted cells.

Culture and analysis of lung-specific gene expression of cord blood-derived CD34⁺ cells. Freshly isolated CD34⁺cells were initially cultured in StemPro-34 Serum-free Medium (SFM) (Invitrogen, Carlsbad, Calif.) supplemented with the following human recombinant factors: stem cell factor (SCF, 100 ng/ml), IL-3 (50 ng/ml) and GM-CSF (25 ng/ml) (all from Miltenyi Biotec). After 72 hours in StemPro-34 SFM expansion medium, the cells were cultured for 1 to 3 weeks in conditions aimed at inducing respiratory epithelial differentiation. These differentiation conditions included small airway growth medium (SAGM) (Samadikuchaksaraei, 2006) (Lonza, Walkersville, Md.), modified mouse tracheal epithelial cell (MTEC) medium, or StemPro-34 SFM supplemented with retinoic acid (RA, Sigma, St. Louis, Mo.) and/or keratinocyte growth factor (KGF, Sigma). Modified MTEC medium consists of MTEC basic medium as described by You et al. (You, 2002), supplemented with 2% NuSerum (Becton-Dickinson), 0.01 μM retinoic acid, hSCF (100 ng/ml), hIL-3 (50 ng/ml) and hGM-CSF (25 ng/ml). In some culture experiments, dexamethasone (DEX) (Sigma) was added to StemPro-34 SFM or modified MTEC medium at concentrations ranging between 10⁻⁵ and 10⁻⁷M (in 0.1% DMSO). Cell viability was assessed by trypan blue exclusion. Morphology of cultured cells was monitored by phase contrast microscopy.

Lung-specific gene expression was assessed by semi-quantitative RT-PCR. Total cellular RNA was extracted from hUCB-CD34⁺ cell lysates using Trizol reagent (Invitrogen) and purified using the RNeasy MinElute Cleanup kit (Qiagen, Valencia, Calif.). Total RNA (1 μg) was reverse-transcribed using the SuperScript III First-Strand Synthesis System (Invitrogen) according to the manufacturer's protocol. Surfactant protein C (SP-C), Clara cell secretory protein (CCSP), aquaporin-5 (AQ-5), thyroid transcription factor-1 (TTF-1), cystic fibrosis transmembrane conductance regulator (CFTR) and glyceraldehyde phosphate dehydrogenase (GAPDH, housekeeping gene) were amplified by polymerase chain reaction (PCR). The primer sequences are listed in Table 1.

TABLE 1

| Gene | Primer Sequences (5′-3′) | Product size (bp) |
|---|---|---|
| SP-C | F: TGG TCC TCA TCG TCG TGG TGA TTG (SEQ ID NO: 1) | 327 |
| | R: CCT GCA GAG AGC ATT CCA TCT GGA AG (SEQ ID NO: 2) | |
| CCSP | F: CTT TCA GCG TGT CAT CGA AA (SEQ ID NO: 3) | 232 |
| | R: TTG AAG AGA GCA AGG CTG GT (SEQ ID NO: 4) | |
| AQP5 | F: CAT CTT CGC CTC CAC TGA CT (SEQ ID NO: 5) | 193 |
| | R: CCC TAC CCA GAA AAC CCA GT (SEQ ID NO: 6) | |
| TTF-1 | F: CCTGTCCCACCTGAACTCC (SEQ ID NO: 7) | 197 |
| | R: CGGCCAGGTTGTTAAGAAAA (SEQ ID NO: 8) | |
| CFTR 1-4 | F: CAG CTG GAC CAG ACC AAT TT (SEQ ID NO: 9) | 160 |
| | R: TTA TCC GGG TCA TAG GAA GC (SEQ ID NO: 10) | |
| GADPH | F: CCC TTC ATT GAC CTC AAC TAC AT (SEQ ID NO: 11) | 407 |
| | R: ACG ATA CCA AAG TTG TCA TGG AT (SEQ ID NO: 12) | |
| ALU | F: 5′-CATGGTGAAACCCCGTCTCTA-3′ (SEQ ID NO: 13) | |
| | R: 5′-GCCTCAGCCTCCCGAGTAG-3′ (SEQ ID NO: 14) | |
| | TaqMan Prove 5′-FAM-ATTAGCCGGGCGTGGTGGCG-TAMRA-3′ | |
| | (SEQ ID NO: 15) | |

Animal husbandry and tissue processing. The previously described lung-specific FasL overexpressing transgenic mouse was used as model for neonatal lung injury/BPD. This model is based on a tetracycline-dependent tet-on overexpression system to achieve time-specific FasL transgene expression in the respiratory epithelium (De Paepe, “Fas-ligand-induced apoptosis of respiratory epithelial cells causes disruption of postcanalicular alveolar development.” Am J Pathol. 2008 July;173(1):42-56). Transgenic (tetOp)₇-FasL mice (“responder line”) were crossed with CCSP−rtTA mice (“activator line”) (Tichelaar J W, Lu W, Whitsett J A: Conditional expression of fibroblast growth factor-7 in the developing and mature lung. J Biol Chem 2000, 275:11858-11864) to yield a mixed offspring of double transgenic (CCSP−rtTA+/(tetOp)₇-FasL+) and single transgenic (CCSP−rtTA+/(tetOp)₇-FasL−) littermates. Upon exposure to the tetracycline analogue, doxycycline (Dox), double transgenic mice exhibit marked pulmonary apoptosis, resulting in BPD-like alveolar disruption; single transgenic littermates remain unaffected and serve as non-injured controls (De Paepe, Am J Pathol. 2008 July;173(1):42-56). Double transgenic mice are denoted in the text as “CCSP+/FasL+” mice, while single transgenic mice are denoted as “CCSP+/FasL−”.

In the studies described herein, Dox (0.01 mg/ml) was added to the drinking water of pregnant and/or nursing dams from E14 to P3 (postnatal day [P]1=day of birth). The progeny (CCSP+/FasL+ and CCSP+/FasL−) were sacrificed at post-transplantation (TPX) day 2 or week 8 by pentobarbital overdose. Lungs were processed as described (De Paepe, Am J Pathol. 2008 July;173(1):42-56). All animal experiments were conducted in accordance with institutional guidelines for the care and use of laboratory animals.

Analysis of apoptotic activity was performed of DOX-treated single transgenic CCSP+/FasL− and double transgenic CCSP+/FasL+ mice on postnatal day 4. TUNEL labeling of lungs of single transgenic pups showed minimal apoptotic activity in distal lung parenchyma. TUNEL labeling of double transgenic littermates showed massive respiratory epithelial apoptosis with accumulation of apoptotic debris in the airspaces.

Intranasal administration of hUCB-CD34+ cells to newborn mice. At P5, hUCB-CD34⁺ cells (5×10⁵ cells/pup) were delivered to Dox-treated double or single transgenic pups by intranasal administration, as described (Fritzell J A, “Fate and effects of adult bone marrow cells in lungs of normoxic and hyperoxic newborn mice.” Am J Respir Cell Mol Biol. 2009 May;40(5):575-87). Freshly isolated hUCB-CD34+ cells were administered to newborn mice immediately after MACS sorting, i.e. within 4 to 5 hours after cord blood harvesting. Sham controls received equal-volume phosphate-buffered saline (PBS) vehicle buffer. The P5 time point was selected as this time point is characterized by marked alveolar epithelial cell apoptotic injury (but diminishing FasL levels) in Dox-treated double transgenic CCSP+/FasL+ mice.

Analysis of homing of hUCB-CD34+ cells in newborn mouse lungs. Homing (for the purpose of the studies described herein, defined as localization of stem cells within distal airways and airspaces) of the intranasally delivered hUCB-CD34+ cells was studied at post-transplantation day 2 by various immunohistochemical techniques. As no anti-human CD34 antibody is commercially available for use in formalin-fixed, paraffin-embedded tissues, we tested a range of candidate antibodies, including anti-human β2-microglobulin, CD31, CD45 and vimentin antibodies, to determine the optimal immunohistochemical method for detection of the distribution of human CD34⁺ cells in murine lungs. Immunohistochemical analysis was by streptavidin-biotin immunoperoxidase method.

Analysis of engraftment of hUCB-CD34+ cells in newborn mouse lungs. Engraftment (for the purpose of this study defined as localization of hUCB-CD34-derived cells within the alveolar wall) was assessed at 8 weeks post-transplantation by fluorescent in situ hybridization (FISH) analysis of formalin-fixed paraffin embedded lung tissues using two types of human chromosome-specific probes. First, the presence of cells of human origin was verified by FISH using chromosome X, Y, and 18 centromere enumeration probes (Vysis, Abbott Laboratories, Abbott Park, Ill.) according to the manufacturer's instructions. The selection of these probes was based on convenience, specifically: their routine availability in a perinatal pathology/cytogenetics service. The tissue sections were coverslipped with mounting media containing DAPI (4′,6-diamidino-2-phenylindole) (Vector Laboratories) and viewed using an epifluorescence microscope equipped with a DAPI/FITC/Texas red triple pass filter set.

Second, FISH analysis was performed with h-alu probes as described by Schormann et al. (Schormann W., “Tracking of human cells in mice.” Histochem Cell Biol. 2008 Aug;130(2):329-38). Briefly, tissue sections were deparaffinized and subjected to epitope retrieval in citrate buffer, pH 6.0. Sections were incubated with fluorescein-labeled alu probe (BioGenex, San Ramon, Calif.) and denatured at 95° C. for 10 min followed by overnight hybridization at 30° C. Following stringent washes and blocking of nonspecific binding sites by a streptavidin-biotin block (Vector Laboratories, Burlingame, Vt.), detection of the human alu probe was achieved using a biotinylated anti-fluorescein antibody (Vector Laboratories) followed by Streptavidin-DyLite 488 (Jackson ImmunoResearch, Baltimore, Md. Finally, the tissue sections were coverslipped with mounting media containing DAPI. Controls for specificity consisted of omission of probe or omission of anti-fluorescein antibody.

The slides were viewed by epifluorescence and confocal microscopy. For confocal microscopy, images were acquired with a Nikon Cl si laser scanning confocal microscope (Nikon, Mellville, N.Y.) using 488 and 561 nm diode lasers. Serial optical sections were acquired separately for each channel with EZ-C1-imaging computer software (Nikon Inc.). Each acquisition was collected with a 60× Plan Apo lens and a scan zoom of 1.78 ×. All images were collected at the same photomultiplier tube settings. Autoquant Deconvolution software was used prior to the assembly of the projections. Projection views, which ranged from 50-120 consecutive single optical sections, were taken at 0.1 μm intervals. NIS Elements AR 3.0 (Nikon Inc.) was used in slice or 3D volume reconstruction and projections.

Engraftment of hUCB-CD34+ cells by FISH analysis was demonstrated using human alu-specific probes. A positive control of human lung showed uniform nuclear alu-FISH positivity. A mouse lung was examined at post-transplantation day 2 and human FITC-positive cells were found to be present in the airspaces, whereas the nuclei of the murine lung tissue were uniformly FISH-negative. Interestingly, FISH-positive nuclear debris, representing nuclear debris from degenerated hUCB-CD34+ cells, was detected in the cytoplasm of several murine cells, presumably, without wishing to be bound or limited by theory, mouse macrophages.

Engraftment of hUCB-CD34+ cells by alu-FISH analysis at post-transplantation week 8 was demonstrated. In some experiments, at least one alu-FISH-positive cell was shown within alveolar septum using confocal microscopy. In some experiments, at least two Alu-FISH-positive cells were shown within alveolar septum using confocal microscopy. In some experiments, at least three Alu-FISH-positive cells were shown within alveolar septum using confocal microscopy.

Analysis of cell fate of hUCB-CD34+ cells in newborn mouse lungs. Analysis of epithelial differentiation. Epithelial differentiation of hUCB-CD34+ cells was assessed by two immunohistochemical methods: streptavidin-biotin immunoperoxidase staining using a human-specific anti-AE1/3 antibody (a pan-cytokeratin antibody) and, in addition, by double immunofluorescence labeling using anti-human vimentin antibody (as marker of human stem cell-derived cells) in combination with anti-human/mouse E-cadherin (as epithelial marker).

Analysis of respiratory epithelial differentiation. In view of the lack of human-specific markers of respiratory epithelial cells that could be used to trace human donor cells in a murine lung background, cell fate mapping was achieved by various combinations of immunofluorescent double labeling. Differentiation of human cord blood-derived CD34+ cells to alveolar type II cells, type I cells or bronchial epithelial Clara cells was assessed by combining anti-human AE1/3 staining with anti-human/mouse prosurfactant protein-C (SP-C, alveolar type II cell marker) (Abcam Inc., Cambridge, Mass.), T1-α (Farr, 1992; Kotton, 2001) (alveolar type I cell marker) (clone 8.1.1, Developmental Studies Hybridoma Bank, Iowa City, Iowa), or Clara Cell Secretory Protein (CCSP, CC-10, bronchial epithelial Clara cell marker) (Upstate Technologies, Lake Placid, N.Y.), respectively. In these immunofluorescence double labeling studies, anti-human AE1/3 antibody was used as a marker of human (i.e. donor-derived) epithelial cells, whereas the cell-specific antibodies were used to provide information about potential respiratory epithelial differentiation of the transplanted cells.

To determine whether donor-derived cells had undergone differentiation to alveolar type I cells, the following approaches were taken: anti-AE1/3 labeling combined with anti- T1-α labeling (Farr A, “Characterization of an antigenic determinant preferentially expressed by type I epithelial cells in the murine thymus.” J Histochem Cytochem. 1992 May; 40(5):651-64; Kotton D N, “Bone marrow-derived cells as progenitors of lung alveolar epithelium.” Development. 2001 December; 128(24):5181-8) (alveolar type I cell marker) (clone 8.1.1, Developmental Studies Hybridoma Bank, Iowa City, Iowa). In addition to standard epifluorescence microscopy, the sections were viewed by confocal microscopy where indicated to ascertain the veracity of co-localization phenomena.

Epithelial differentiation of engrafted hUCB-CD34+ cells using e-cadherin was demonstrated. Combined anti-human vimentin (Alexafluor 488) and anti-human/mouse e-cadherin (Cy3) immunofluorescence showed colocalization of both signals in cells within the alveolar wall, confirming the epithelial differentiation of engrafted human-derived cells. In this stain, vimentin was used as marker of cells of human derivation, while e-cadherin was used as an epithelial marker. Confocal microscopy was performed using an original magnification of ×1800.

Alveolar type II cell differentiation of engrafted hUCB-CD34+ cells was demonstrated in single transgenic mice at post-transplantation week 8. Combined anti-human cytokeratin (Alexafluor 488) and anti-human/mouse SP-C (Cy3) immunofluorescence was shown in a cell within the alveolar wall using confocal microscopy. In non-injured lungs, differentiation of human-derived epithelial cells to alveolar type II cells was a rare occurrence.

Alveolar type II cell differentiation of engrafted hUCB-CD34+ cells using double transgenic mice at post-transplantation week 8 was demonstrated. Combined anti-human cytokeratin (Alexafluor 488) and anti-human/mouse SP-C (Cy3) immunofluorescence in a cell within the alveolar wall was shown using confocal microscopy. Differentiation of human-derived epithelial cells to alveolar type II cells was significantly more frequent in injured lungs. Relatively abundant surfactant protein was present in human-derived cells, in cellular distribution similar to that of native murine cells, including juxtamembranous location indicative of secretory activity.

Alveolar type II cell differentiation of engrafted hUCB-CD34+ cells was demonstrated using a double transgenic mouse at post-transplantation week 8. Combined anti-human cytokeratin (Alexafluor 488) and anti-human/mouse SP-C (Cy3) immunofluorescence using confocal microscopy was shown. Presence of granular surfactant-immunoreactive material was frequently observed in cells adjacent to human-derived alveolar type II cells, consistent with the appearance of intermediate forms normally created during the generation of type I cells from type II cells by asymmetric division. In addition, human cytokeratin-immunoreactive material was observed in these cells neighboring human-derived type II cells, indicating that these cells were derived from human type II cells.

Alveolar type II cell differentiation of engrafted hUCB-CD34+ cells using a double transgenic mouse at post-transplantation week 8 was demonstrated. Combined anti-human cytokeratin (Alexafluor 488) and anti-human/mouse SP-C (Cy3) immunofluorescence using confocal microscopy was shown. The presence of granular surfactant-immunoreactive material as well as human cytokeratin-immunoreactive material in cells adjacent to human-derived alveolar type II cells, was consistent with the appearance of intermediate forms (with phenotype intermediate between type I and type II cells) created during the generation of type I cells from type II cells by asymmetric division.

Alveolar type II cell differentiation of engrafted hUCB-CD34+ cells using a double transgenic mouse at post-transplantation week 8 was demonstrated. Combined anti-human cytokeratin (Alexafluor 488) and anti-human/mouse SP-C (Cy3) immunofluorescence using confocal microscopy was shown. Human-derived alveolar type II cells that could be observed in the process of cell division further demonstrated human-derived alveolar type II cells functioning as progenitor cells of the distal respiratory unit (type I and type II cells).

Alveolar type I cell differentiation of engrafted hUCB-CD34+ cells using a double transgenic mouse at post-transplantation week 8 was demonstrated. Combined anti-human cytokeratin (Alexafluor 488) and anti-human/mouse T1α (Cy3) immunofluorescence using confocal microscopy was shown. T1α is a membrane-associated marker of alveolar epithelial type I cells. Human cytokeratin-positive cells were noted deeply engrafted within the alveolar wall and enveloped by type I cell extensions. Colocalization of human cytokeratin and T1α was noted, indicative of human derivation of type I cells surrounding human-derived type II cells.

Analysis of proliferation of hUCB-CD34-derived cells in newborn mouse lungs. Analysis of proliferative activity of engrafted hUCB-CD34-derived cells was assessed by combining alu-FISH analysis with anti-Ki67 immunohistochemistry. To this end, alu-FISH analysis was followed by incubation of the tissue sections with anti-human Ki67 antibody) followed by Cy3-labeled secondary antibody.

The proliferative activity of hUCB-CD34-derived epithelial cells was assessed by double immunofluorescence labeling using anti-human AE1/3 antibody (as marker of human-derived epithelial cells) in combination with anti-Ki67 antibody using methods previously described.

Proliferation of engrafted hUCB-CD34-derived epithelial cells using a double transgenic mouse at post-transplantation week 8 was demonstrated. Combined anti-human cytokeratin (Alexafluor 488) and anti-human/mouse Ki67 (Cy3) immunofluorescence using confocal microscopy was shown. Proliferating human-derived epithelial cells, non-proliferating human-derived epithelial cells, and several proliferating murine lung cells were observed. Proliferative activity of stably engrafted human-derived epithelial cells 8 weeks after transplantation was demonstrated.

Analysis of effect of hUCB-CD34+ cells on lung growth and alveolarization. Body weight was determined at the time of sacrifice. Morphometric assessment of growth of peripheral air-exchanging lung parenchyma and contribution of the various lung compartments (airspace versus parenchyma) to the total lung volume was performed using standard stereological volumetric techniques, as described elsewhere. Alveolarization was quantified by computer-assisted histomorphometric analysis of the mean cord length (MCL), as described (De Paepe, Am J Pathol. 2008 July;173(1):42-56). All morphometric assessments were made on coded slides by a single observer who was unaware of the experimental condition or genotype of the animal analyzed.

Data analysis. Values are expressed as mean±standard deviation (SD) or, where appropriate, as mean±standard error of the mean (SEM). The significance of differences between groups was determined with the unpaired Student's t-test or ANOVA with post-hoc Scheffe test where indicated. The significance level was set at P<0.05. Statview software (Abacus, Berkeley, Calif.) was used for all statistical work.

Intraperitoneal administration of hUCB-CD34+ cells. In some experiments, freshly isolated or expanded and differentiated hUCB-CD34+ cells were administered to Dox-treated double or single transgenic pups at P5 by intraperitoneal route to achieve systemic, rather than intranasal/intratracheal cell delivery. To this end, cells (5×10⁵ hUCB-CD34+ cells or 1×10⁶ CD34-derived cells in 25 μl sterile PBS) were injected through a tuberculin syringe into the left lower quadrant. Sham controls received equal-volume phosphate-buffered saline (PBS) vehicle buffer.

Engraftment of hUCB-CD34-derived cells at 8 weeks after intraperitoneal administration was studied by FISH analysis using alu probes. FISH analysis of lung sections 8 weeks after intraperitoneal transplantation of hUCB-CD34+ cells revealed scattered alu-positive nuclei, distributed evenly in both lungs. In most areas, alu-positive cells appeared to be single. However, multiple high power fields contained two or more alu-positive nuclei. The nuclear shape ranged from curved to oval or round. The alu-positive nuclei were localized to alveolar septa. To determine whether lung injury influenced engraftment rates, we determined the density of alu-positive cells in Dox-treated double versus single transgenic animals. As in intranasally delivered cells, lung injury at time of administration did not affect engraftment efficiency following systemic delivery.

Engraftment of intraperitoneally administered hUCB-CD34+ cells by alu-FISH analysis at post-transplantation week 8 was demonstrated. Alu-FISH positive cells within alveolar septa were observed using confocal microscopy, and curvilinear shape and location within secondary crest indicated endothelial differentiation.

Arming of hUCB-CD34+ cells with hCD34 ×mVCAM-1 bispecific antibodies. In some experiments freshly isolated (or ex vivo expanded) hUCB-CD34+ cells were armed with bispecific hCD34×mVCAM-1 antibodies in order to target hUCB-CD34+ cells to pulmonary epithelium and endothelium following intranasal and intraperitoneal delivery, respectively. Pulmonary endothelium and, to lesser extent, epithelium, expresses VCAM-1, especially under conditions of injury.

To develop a new hCD34×mVCAM-1 bispecific antibody (BiAb), heteroconjugation was performed by Dr. L. Cousens, according to published methods. Briefly, anti-human CD34 antibody is reacted with Traut's reagent (2-iminothiolane HC1). In parallel, anti-murine-VCAM-1 antibody or an isotype-matched (negative) control antibody is reacted with sulphosuccinimidyl 4-(N-maleimidomethyl) cyclohexane-l-carboxylate (Sulpho-SMCC). Unbound cross-linker is removed by antibody purification on PD-10 columns. Cross-linked antibodies are then immediately mixed at equimolar concentrations and conjugated overnight.

To validate the integrity, specificity and activity of new hCD34×mVCAM-1 BiAb in vitro, the heteroconjugation products are resolved by SDS-PAGE and detected by Gelcode Blue staining. Densitometric quantitation is performed on the separated products allowing estimation of the proportion of monomer (unconjugated mAbs; inactive), dimer (heteroconjugated products comprised of one cell-specific mAb pair; active), and multimer (products comprised of more than one heteroconjugated mAb pair; active) fractions. Monomer versus dimer ratios is determined by SDS-PAGE and image analysis of the resulting gel. Dose-dependent binding of BiAb to cells is established by flow cytometry, a method based on BiAbs being designed with 2 distinct isotypes. Specifically, the CD34 antibody is a rat IgG2b and the VCAM-1 antibody is a rat IgG2a isotype. Consequently, when BiAbs bound to CD34 (rat IgG2b)+stem cells, are detected with fluorochrome-conjugated secondary antibodies specific for rat IgG2a, it indicates that a heteroconjugated product has bound to the cell. Human UCB-CD34+ cells are incubated with 0-1000 ng hCD34×mVCAM-1 BiAb per 10⁶ cells and washed to remove unbound BiAb. BiAb binding (a.k.a. “arming”) is detected by staining with goat anti-rat IgG2a-FITC and analyzed by flow cytometry.

The functional ability of BiAbs is tested in vitro by assessing the ability of BiAb-armed cell populations to aggregate when co-incubated with an immobilized antigen source. To test the function of the CD34×VCAM-1 BiAb, human UCB CD34+ cells are armed with BiAb and their ability to aggregate on monolayers of VCAM-1-expressing murine lung epithelial cells (MLE-12) is tested, similar to published methods. When successful heteroconjugation of a BiAb product has been validated for specificity and activity, an isotype-matched control BiAb is produced, consisting of the cell-specific antibody heteroconjugated to an antibody heteroconjugated to an antibody isotype-matched to the tissue-specific antibody of an irrelevant specificity. Monitoring the in vivo trafficking and engraftment of human CD34+ cells, armed with new hCD34×mVCAM-1 BiAbs, following intranasal delivery to newborn mice.

Human UCB-CD34+ cells, armed with CD34×VCAM-1 antibody, are traced following intranasal delivery at determined doses. Normoxic and hyperoxia-exposed newborn mice receive BiAb or control antibody-armed UCB-CD34+ cells at P4 at determined doses. Mice are euthanized 2 days or 2 wks later. Engrafted human cells are detected and localized by immunohistochemistry using anti-human epitope-specific antibodies and human-specific FISH analysis. In some embodiments, BiAb-armed cells are fluorescently labeled with carboxyfluorescein diacetate succinimidyl ester (CFSE) and subsequently tracked with confocal or epifluorescent microscopy. Engrafted human cells are quantitated by qPCR for human Alu sequences as well as anti-human cell-specific immunohistochemistry combined with stereological volumetry.

The hCD34×mVCAM-1 antibodies were prepared for this purpose by Dr. L. Cousens (Roger Williams Medical Center). The integrity, specificity and activity of the hCD34×mVCAM-1 BiAb was validated by Dr. Cousens in vitro.

To arm the cells, hUCB-CD34+ cells (either freshly isolated or after 3-day expansion when still >95% of cells were still CD34+ by FACS and immunohistochemistry) were incubated with the hCD34×mVCAM-1 BiAb (1000 ng per 106 cells) and washed to remove unbound BiAb. BiAb binding (also defined herein as “arming” was detected by staining with goat anti-rat IgG2a-FITC and analyzed by flow cytometry.

To investigate the SDF-1/CXCR4 axis in homing/engraftment of hUCB-CD34+ cells following intranasal delivery, surface CXCR4 expression in UCB cells at various steps of preparation (UCB-MNC, UCB-CD34+ before and after culture) by flow cytometry is determined using phycoerythrin-labeled anti-human CXCR4 antibody (Pharmingen). In addition, the spatiotemporal patterns of SDF-1 expression in hyperoxic and normoxic newborn lungs are studied by ELISA and immunohistochemistry. Homing/engraftment efficiency of hUCB-CD34+ cells following inhibition or stimulation of the SDF-1/CXCR4 axis are then determined. The effects of CXCR4 neutralization by preincubation with anti-human CXCR4 antibody (mAb 12G5, R&D Systems Inc.), which inhibits CXCR4/SDF-1 interactions as well as the effects of in vivo administration of SDF-1 (SDF1, PeproTech Inc.), which enhances their activity are tested. The homing and engraftment efficiencies are determined at post-transplant days 2 and week 2, using immunohistochemical and molecular methods.

Engraftment of intraperitoneally administered hUCB-CD34+ cells armed with hCD34 X mVCAM-1 bispecific antibodies was demonstrated by Alu-FISH analysis at post-transplantation week 8 using confocal microscopy. Several alu-FISH positive cells were found within alveolar septa. Doublets were indicative of recent replication.

Isolation of CD34+ Cells from Human Cord Blood.

Human umbilical cord blood was obtained from uncomplicated full-term cesarean deliveries (n=47) at Women and Infants Hospital according to protocols approved by the Institutional Review Board. Cord blood (CB) was collected in citrate phosphate dextrose whole blood collection bags (Baxter Healthcare Corp., Deerfield, Ill.) and processed within 2 hours after delivery. Mononuclear cord blood cells (CB-MNCs) were isolated by Ficoll-Hypaque density gradient centrifugation (Fisher BioReagents, Pittsburgh, Pa.). CB-CD34+ cells were isolated from mononuclear cell suspensions by immunomagnetic cell sorting (MACS) using anti-human CD34 microbeads according to the manufacturer's instructions (Miltenyi Biotec, Bergisch Gladbach, Germany)^{52, 53}. CD34+ cell purity was determined by immunocytochemistry and flow cytometry analysis of the MACS product using a phycoerythrin-conjugated anti-human CD34 antibody (130-081-002, Miltenyi Biotec). Cell viability was determined by Trypan blue exclusion. Cell purity and viability were studied in eight randomly selected cell preparations. Animal husbandry and tissue processing.

The previously described lung-specific FasL overexpressing transgenic mouse^{8,51} was used as model for neonatal lung injury/BPD. This model is based on a tetracycline-dependent tet-on overexpression system to achieve time-specific FasL transgene expression in the respiratory epithelium⁸. Transgenic (tetOp)₇-FasL mice (“responder line”) were crossed with CCSP−rtTA mice (“activator line”) (kindly provided by Dr. J. Whitsett, University of Cincinnati, Ohio)⁵⁴ to yield a mixed offspring of double transgenic (CCSP−rtTA+/(tetOp)₇-FasL+) and single transgenic (CCSP−rtTA+/(tetOp)₇-FasL−) littermates. Upon exposure to the tetracycline analogue, doxycycline (Dox), double transgenic mice (CCSP+/FasL+) exhibit marked pulmonary apoptosis, resulting in BPD-like alveolar disruption; single transgenic littermates (CCSP+/FasL−) remain unaffected and serve as non-injured controls⁸. The transgenic animals are generated in a FVB/N genetic background and have an intact immune system.

In this study, Dox (0.01 mg/ml) was added to the drinking water of pregnant and/or nursing dams from embryonal day 14 (E14) to postnatal day 3 (P3; postnatal day P1=day of birth). The progeny (both CCSP+/FasL+ and CCSP+/FasL−) were sacrificed at post-inoculation day 2 or week 8 by pentobarbital overdose. Between five and ten animals of each genotype and treatment group were studied at each time point. Lungs were processed as described⁸. All animal experiments were conducted in accordance with institutional guidelines for the care and use of laboratory animals. Intranasal administration of CB-CD34+ cells to newborn mice.

At postnatal day 5 (P5), CB-CD34+ cells (1 to 5×10⁵ cells/pup) were delivered to Dox-treated double or single transgenic pups by intranasal administration, as previously described²⁴. Freshly isolated CB-CD34+ cells, derived from eight different cord blood cell preparations, were administered to newborn mice immediately after MACS sorting, i.e. within 4 to 5 hours after cord blood harvesting. Sham controls received equal-volume phosphate-buffered saline (PBS) vehicle buffer. Intranasal inoculation was performed at P5 as this time point is characterized by marked alveolar epithelial cell apoptotic injury and remodeling in Dox-treated double transgenic CCSP+/FasL+ mice.

### Analysis of Engraftment of CB-CD34+ Cells in Newborn Mouse Lungs.

Delivery of the intranasally administered CB-CD34+ cells to distal airways and airspaces was studied at post-inoculation day 2 by anti-human vimentin (N1521, DAKO, Glostrup, Denmark) immunohistochemistry. Antibody binding was detected by streptavidin-biotin immunoperoxidase method. Long-term engraftment of cord blood-derived cells was assessed at 8 weeks post- inoculation. The presence of human-derived cells was assessed by real-time PCR (qRT-PCR) analysis of human alu sequences, according to methods described by McBride et al.⁵⁵ (Table 1). Genomic DNA was extracted from whole lung lysates using Wizard Genomic DNA Purification Kit (Promega Corporation, Madison, Wis.). Standard curves were generated by serially diluting human genomic DNA prepared from cord blood cells into murine genomic DNA using a total of 50 ng DNA per reaction.

In addition, the distribution of engrafted cells was studied by fluorescent in situ hybridization (FISH) analysis of formalin-fixed paraffin-embedded lung tissues using two types of human chromosome-specific probes. The presence of cells of human origin was verified by multicolor FISH analysis using chromosome X, Y, and 18 centromere enumeration probes (Vysis, Abbott Laboratories, Abbott Park, Ill.) according to the manufacturer's instructions. The selection of these probes was based on their routine successful application in our perinatal pathology/cytogenetics service. The tissue sections were coverslipped with mounting media containing DAPI (4′,6-diamidino-2-phenylindole) (Vector Laboratories) and viewed using an epifluorescence microscope equipped with a DAPI/FITC/Texas red triple pass filter set.

FISH analysis with human-specific alu probes was also performed as described by Schormann et al.⁵⁶. Briefly, tissue sections were deparaffinized and subjected to epitope retrieval in citrate buffer, pH 6.0. Sections were incubated with fluorescein-labeled alu probe (PR-1001-01, BioGenex, San Ramon, Calif.) and denatured at 95° C. for 10 min followed by overnight hybridization at 30° C. Following stringent washes and blocking of nonspecific binding sites by a streptavidin-biotin block (Vector Laboratories, Burlingame, Vt.), detection of the human alu probe was achieved using a biotinylated anti-fluorescein antibody (Vector Laboratories) followed by Streptavidin-DyLite 488 (Jackson ImmunoResearch, Baltimore, Md.). Controls for specificity consisted of omission of probe or omission of anti-fluorescein antibody, which abolished all staining. The slides were viewed by confocal microscopy, as previously described²⁴.

### Analysis of Cell Fate of CB-CD34+ Cells in Newborn Mouse Lungs.

Analysis of epithelial and respiratory epithelial differentiation. The epithelial differentiation of CB-CD34+ cells was assessed by streptavidin-biotin immunoperoxidase staining using a human-specific antibody against cytokeratin (M3515, AE1/3, DAKO). In view of the lack of human-specific markers of respiratory epithelial cells that could be used to trace human donor cells in a murine background, further cell fate mapping was achieved by combinations of immunofluorescent double labeling. In the double labeling studies, anti-human cytokeratin antibody was used as a marker of human (i.e. cord blood-derived) epithelial cells, whereas the cell-specific antibodies were used to provide information about potential respiratory epithelial differentiation of the cord blood-derived engrafted cells.

Differentiation of human cord blood-derived CD34+ cells to alveolar type II cells was assessed by combining anti-human cytokeratin staining with anti-prosurfactant protein-C (SP-C) (ab28744, Abcam Inc., Cambridge, Mass.). To study differentiation of donor-derived cells to alveolar type I cells, anti-human cytokeratin staining was combined with anti-T1alpha (podoplanin) labeling57,58 (clone 8.1.1, Developmental Studies Hybridoma Bank, Iowa City, Iowa). Differentiation of human cord blood-derived CD34+ cells to bronchial epithelial Clara cells was assessed by combining anti-human cytokeratin staining with Clara Cell Secretory Protein (CCSP, CC-10) labeling (07-623, Upstate Technologies, Lake Placid, N.Y.). All sections were viewed by confocal microscopy.

Analysis of proliferation. The proliferative activity of engrafted cord blood-derived cells was assessed by combining human alu-FISH analysis with anti-Ki67 immunohistochemistry. To this end, human alu-FISH analysis was performed as described above, followed by incubation of the tissue sections with rabbit monoclonal anti-Ki67 antibody (4203-1, Epitomics, Burlingame, Calif.), biotinylated anti-rabbit secondary antibody (Vector Laboratories) and, finally, AlexaFluor 594-Streptavidin Conjugate (Vector Laboratories). Similarly, the specific proliferative activity of cord blood-derived epithelial cells was assessed by double immunofluorescence labeling using anti-human cytokeratin antibody in combination with anti-Ki67 labeling, using previously described methods⁴.

Analysis of fusion. Previous studies, based on heart and liver transplant models, have suggested that the presence of donor-derived differentiated cells may be attributable, at least in part, to fusion of donor-derived stem or progenitor cells with mature recipient cells. Most well-described models of xenogeneic human to mouse transplantation in which fusion occurs, such as fusion of CB-CD34+ cells (or their progeny) with murine hepatocytes, are characterized by nuclear fusion as demonstrated by colocalization of donor (human) and recipient (murine) genome in the same nucleus⁵⁹⁻⁶¹. To investigate the occurrence of cellular fusion, FISH analysis with human chromosome-specific probes was combined with FISH analysis using mouse chromosome-specific probes (Pancentromeric Mouse Chromosome Paint, 1697-Mcy3-02, Cambio Ltd., Cambridge, UK). Sections were processed for alu-FISH analysis as described above with a single modification: at the time of hybridization, tissues were incubated simultaneously with human alu probes and Cy3-labeled pancentromeric mouse probes.

Data analysis

Values are expressed as mean±standard deviation (SD). The significance of differences between groups was determined with the unpaired Student's t-test or ANOVA with post-hoc Scheffe test where indicated. The significance level was set at P<0.05. Statview software (Abacus, Berkeley, Calif.) was used for all statistical work.

### Introduction

Premature infants treated with supplemental oxygen and mechanical ventilation are at risk for bronchopulmonary dysplasia (BPD), or chronic lung disease of the preterm newborn, a complex condition characterized by an arrest of alveolar development¹. Although surfactant therapy, antenatal steroids, and changes in neonatal intensive care have modified its phenotype, BPD remains a significant complication of premature birth. The main pathological hallmark of BPD is an arrest of alveolar development, characterized by large and simplified distal airspaces²⁻⁴. In addition, several reports have shown that the lungs of ventilated preterm infants with early BPD show markedly increased levels of alveolar epithelial cell death⁵⁻⁷. We recently demonstrated that increased alveolar epithelial apoptosis induced by Fas-ligand overexpression in newborn mice is sufficient to disrupt alveolar remodeling⁸, indicating that the loss of alveolar epithelial cells plays a critical role in the arrested alveolar development seen in BPD. Accordingly, as shown herein, cell-based therapies aimed at restoring or protecting the alveolar epithelium in injured newborn lungs can be beneficial.

Some publications over the past decade have suggested that bone marrow-derived stem and progenitor cells can structurally engraft as mature differentiated airway and alveolar epithelial cells⁹⁻¹⁹ [reviewed in²⁰]. However, the field has had conflicting results. Epithelial engraftment is suggested by some investigators to be a rare event, regardless of the marrow-derived cell type used or the type of antecedent lung injury [reviewed in²⁰]. In fact, it has recently been questioned whether engraftment and transdifferentiation can occur at all, based on failure to duplicate these results using state-of-the-art morphological techniques²¹⁻²⁴. Though several studies have reported seemingly unequivocal engraftment of donor-derived airway and/or alveolar epithelium following adult stem cell administration^{10,18,25-27} functional reconstitution by and clonal expansion of the engrafted cells have not yet been demonstrated.

In addition, while heavy experimental emphasis has been placed on marrow-derived stem cell therapies, little is know about the potential role of non-marrow-derived stem cells, such as those derived from umbilical cord blood. Human umbilical cord blood is a readily available source of autologous hematopoietic stem cells, endothelial cell precursors, mesenchymal progenitors, and multipotent/pluripotent lineage stem cells²⁸⁻³². Cord blood stem cells can be collected at no risk to the donor, have low immune reactivity, low inherent pathogen transmission, and are not subject to the social and political controversy associated with embryonic stem cells. Cord blood stem cells are particularly attractive in the newborn context where the infant's own cord blood-derived stem cells could be used as an autologous transplant.

Cord blood stem cells can be induced to differentiate along neural, cardiac, epithelial, hepatic, pancreatic and dermal pathways³³⁻⁴⁴. The role of cord blood-derived stem cells in lung repair remains largely unexplored. Recent studies have shown that cord blood-derived mesenchymal stem cells can decrease lung injury and/or promote tissue repair after lung injury, even without significant engraftment as lung epithelial cells^{9,45,46}. The mechanisms underlying these mesenchymal stem cell-associated beneficial effects are not fully determined, but are believed to be related, without wishing to be bound or limited by theory, to anti-inflammatory paracrine factors⁴⁷. While the use of cord blood- or bone marrow-derived mesenchymal stem cells may lead to invaluable therapeutic strategies for older patients with end-stage lung disease, caution may be warranted before their use in younger age groups can be considered. Mesenchymal stem cells continue to be poorly characterized and not uniformly defined, compromising interpretation and comparison of results obtained in different laboratories. More ominously, there is increasing clinical and experimental evidence suggesting that mesenchymal stem cells may undergo malignant transformation and give rise to sarcomatous neoplasms⁴⁸⁻⁵⁰. This diminishes the enthusiasm for use of these stem cells as therapeutic modality in young children. Accordingly, described herein are methods of treatment that do not use mesenchymal stem cells.

In contrast to mesenchymal stem cells, hematopoietic progenitor cells are better and more uniformly characterized, are more easily isolated, and have an excellent and long-standing safety record after decades of use in clinical transplantation. The aim of the present study was to determine, using state-of-the-art morphologic techniques, whether human cord blood-derived CD34+ hematopoietic progenitor cells have the capacity to 1) engraft in injured newborn lungs, 2) undergo functional differentiation to respiratory epithelial cells, and 3) regenerate injured lung epithelium. As in a previous study²⁴, the intranasal/intrapulmonary route of administration was chosen, rather than the systemic route for delivery of stem cells. The direct intrapulmonary delivery of stem cells represents a biologically more sound strategy for restoration of the respiratory epithelium²⁴.

Furthermore, the intrapulmonary route is highly clinically relevant. As many preterm infants are intubated, intrapulmonary delivery via the endotracheal tube is within the scope of the current practice of administration of exogenous surfactant and antioxidants in some embodiments.

As a model of neonatal lung injury, newly generated conditional respiratory epithelium-specific Fas-ligand (FasL) overexpressing transgenic mouse were used^{8,51}. When FasL overexpression is targeted to the perinatal period, this apoptosis-induced transgenic mouse model provides a faithful replication of both the early apoptotic injury and subsequent alveolar simplification typical of preterm infants with BPD^{8,51}.

### Results

Harvesting of CD34+ Cells from Umbilical Cord Blood.

Umbilical cord blood was collected from 47 uncomplicated full-term cesarean deliveries. The average cord blood collection volume was 92.4±32.0 ml (range: 39 to 191 ml). Following Ficoll gradient centrifugation, cord blood-derived (CB) mononuclear cells were subjected to immunomagnetic sorting (MACS) by positive selection using a CD34 MicroBead Kit (Miltenyi Biotec). On average, 1.7±1.2×10⁶ CB-CD34+ cells were isolated per placenta (range: 0.2 to 4.5×10⁶). The CD34+ cell yield per unit of cord blood volume varied greatly between cases and ranged between 0.24 and 3.66×10⁶ CD34+ cells per 100 ml cord blood (average: 1.52±0.95×10⁶ CD34+ cells per 100 ml). CD34+ cell purity was greater than 95%, as determined by flow cytometry analysis and immunohistochemical analysis of cytospin preparations using FITC-labeled anti-CD34 antibodies (not shown). Cell viability after Ficoll centrifugation and MACS sorting, determined by trypan blue exclusion, was >92%.

Analysis of early Distribution of CB-CD34+ Cells in Lungs of Newborn Mice following Intranasal Administration.

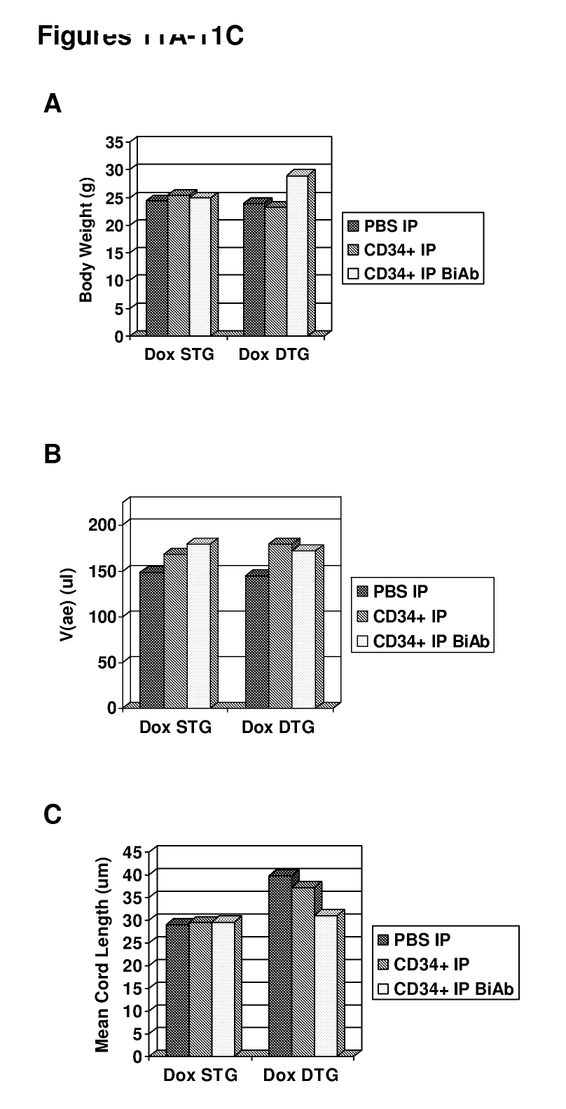

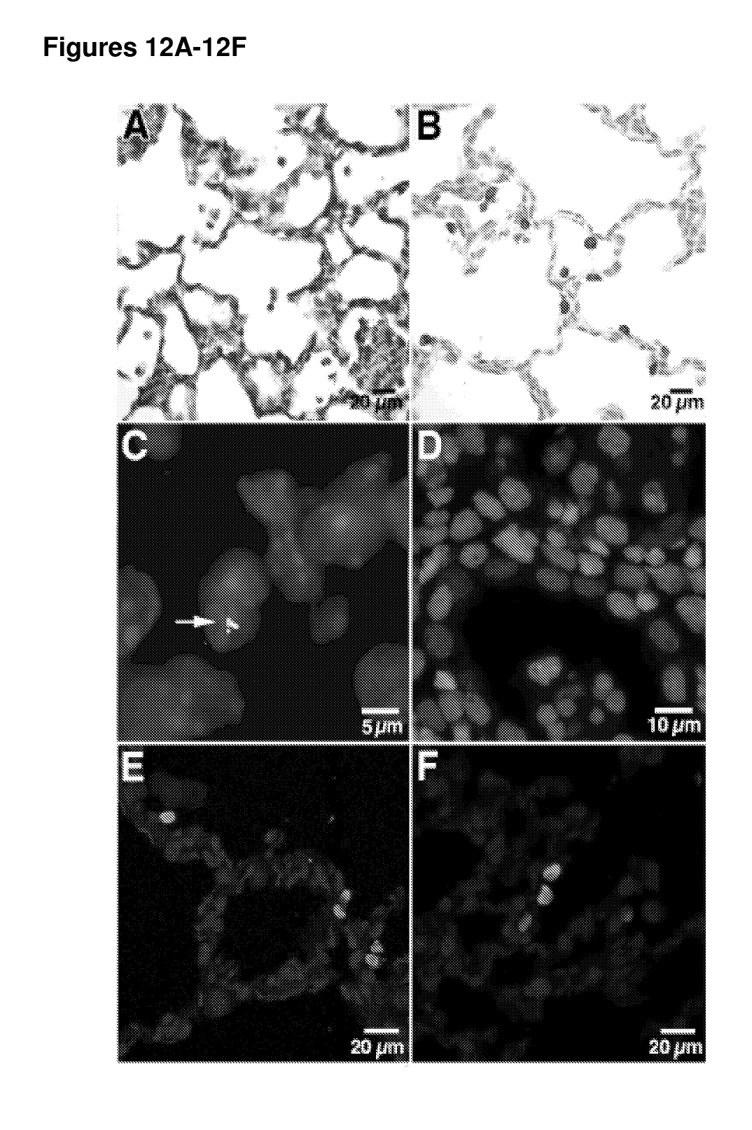

Delivery of intranasally administered CB-CD34+ cells to distal airways and airspaces was monitored by anti-human vimentin immunohistochemistry at post-inoculation day 2. Intranasal administration of CB-CD34+ cells in newborn mice resulted in even and effective cellular distribution in both lungs (FIGS. 12A-12B), confirming our previous results with murine whole bone marrow cells^{'}. Focal intraalveolar macrophage collections were noted in association with degenerating donor-derived cells (FIG. 12A). Intraalveolar inflammatory aggregates and associated cellular debris appeared to be more prevalent in double transgenic recipients. There was no histopathologic evidence of interstitial inflammation. Omission of primary anti-vimentin antibody abolished all immunoreactivity. Anti-human vimentin staining of lungs of control newborn mice that did not receive CB-CD34+ cells was negative. In initial experiments, we performed a survey of human vimentin immunoreactivity in other organs. No human vimentin-immunoreactive cells were detected in liver, spleen, bone marrow or kidneys, suggesting the early distribution of intranasally delivered donor cells was confined to the lungs. As previously reported²⁴ , intranasal delivery was associated with cell loss in the gastrointestinal tract, as demonstrated by the occasional presence of human vimentin-positive cells in the stomach.

### Analysis of Long-Term Engraftment of CB-CD34+ Cells in Lungs of Newborn Mice.

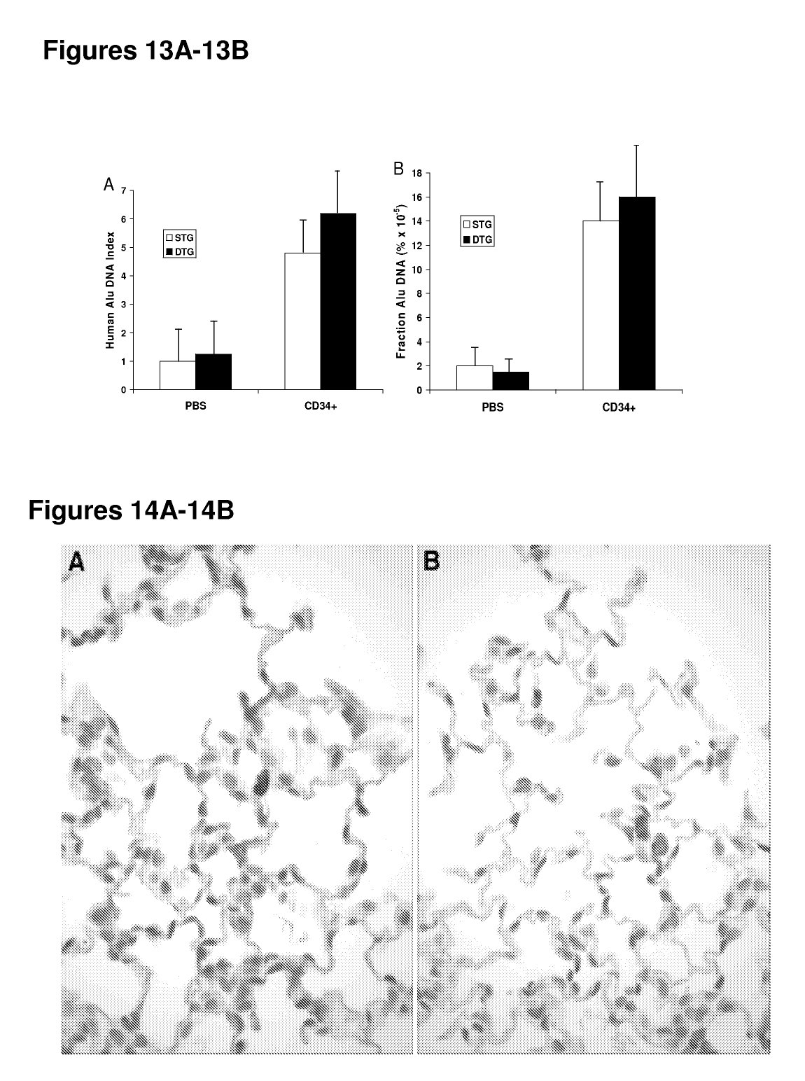

Real-time qRT-PCR analysis of lung lysates at post-inoculation week 8 revealed the presence of human alu DNA sequences in all recipient lungs, albeit at low levels (FIGS. 13A-13B). The amount of human alu DNA recovered from lung homogenates varied greatly between animals but was comparable overall between single and double transgenic recipients: both the Alu DNA Index (amount of Alu PCR amplification product in lungs of CB-CD34+ recipients versus lungs of PBS-treated animals) and the fraction of human genomic DNA relative to total lung DNA were similar in both groups (FIGS. 13A-13B).

Two types of fluorescence in situ hybridization (FISH) analysis to identify human-derived engrafted cells were used. FISH analysis using human chromosome X, Y, and 18 centromere enumeration probes detected scattered human-derived cells in the alveolar septa (FIG. 12C). However, interpretation of the results of conventional FISH analysis using color-coded centromeric probes was often hindered by the small size of the signal and high background noise. FISH analysis using human alu probes resulted in easily detectable and highly specific labeling of human-derived cells (FIGS. 12D-12F), and thus allowed more reliable interpretation and quantitation of results. Alu-positive nuclei were readily identified in all transplanted animals (FIGS. 12E-12F). The engrafted cells were evenly distributed in central and peripheral lung parenchyma without obvious geographic predilection. While the majority of human cells were single, occasional aggregates of alu-positive cells were identified within the same microscope field and in a paired or contiguous pattern, suggestive of clonogenic expansion (FIGS. 12E-12F).

To determine the effect of lung injury on the recovery rates of cord blood-derived cells at 8 weeks post-inoculation, the density of alu-FISH positive nuclei in Dox-treated double transgenic animals with that in single transgenic animals wascompared. The density of alu-positive cells was similar in both groups (5.6±1.3 cells per 10 high power fields in double transgenic animals versus 4.7±1.4 cells per 10 high power fields in single transgenic animals; 4 animals per group). Taken together, the qRT-PCR and alu FISH data indicate that intranasal inoculation of human CB-CD34+ cells in newborn mice results in long-term, stable pulmonary engraftment of cord blood-derived cells, both in injured and non-injured lungs.

### Analysis of Cell Fate of CB-CD34+ Cells in Lungs of Newborn Mice.

Analysis of epithelial differentiation. The potential of CB-CD34+ cells (or their progeny) to undergo epithelial differentiation by anti-human cytokeratin immunohistochemical analysis of lung tissues at 8 weeks post-inoculation was next assessed. Scattered, and occasionally clustered, human cytokeratin-positive cells were readily detected in all lungs (FIGS. 14A-14B). Some human cytokeratin-positive cells were large and ovoid or spherical in shape with ample cytoplasm. Others appeared more elongated and aligned with the alveolar wall (FIGS. 14A-14B). As demonstrated by the lack of staining in murine alveolar and bronchial epithelial cells, the anti-human cytokeratin monoclonal antibody proved to be specific for epithelial cells of human origin (FIGS. 14A-14B). Omission of primary antibody abolished all staining

Analysis of respiratory epithelial differentiation. The above results demonstrate that intranasally delivered CB-CD34+ cells are capable of undergoing epithelial differentiation. To determine their capacity to undergo respiratory epithelial differentiation, double immunofluorescence labeling studies combining anti-human cytokeratin staining (as marker of human-derived epithelial cells) with cell-specific respiratory or airway epithelial markers was performed.

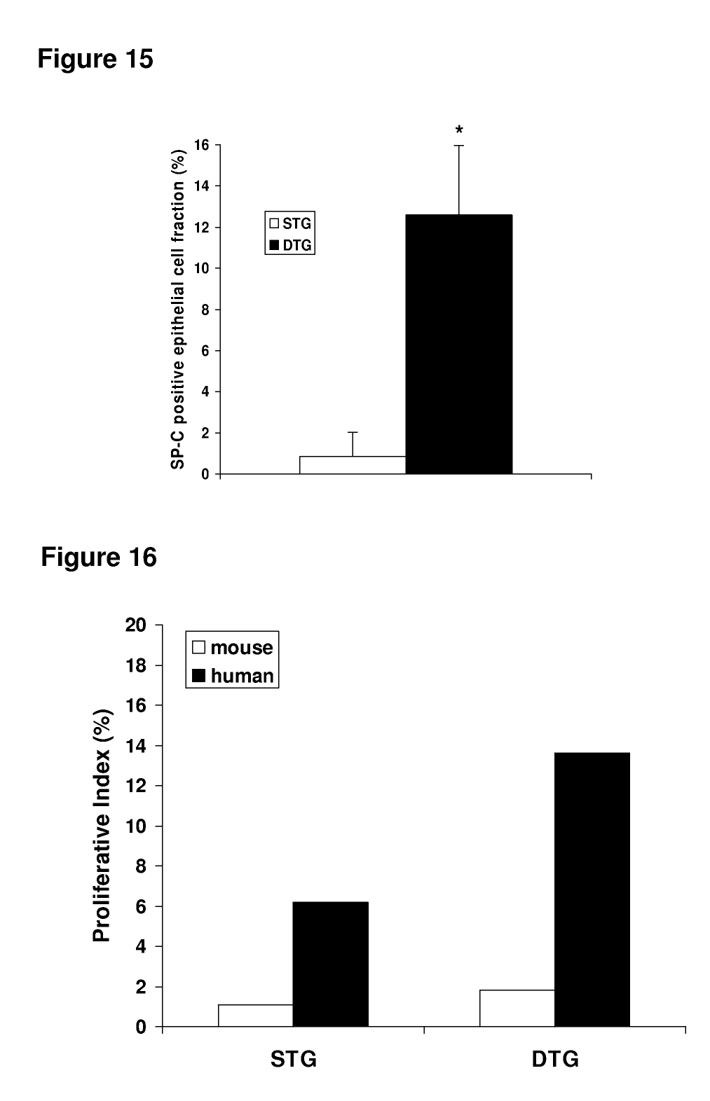

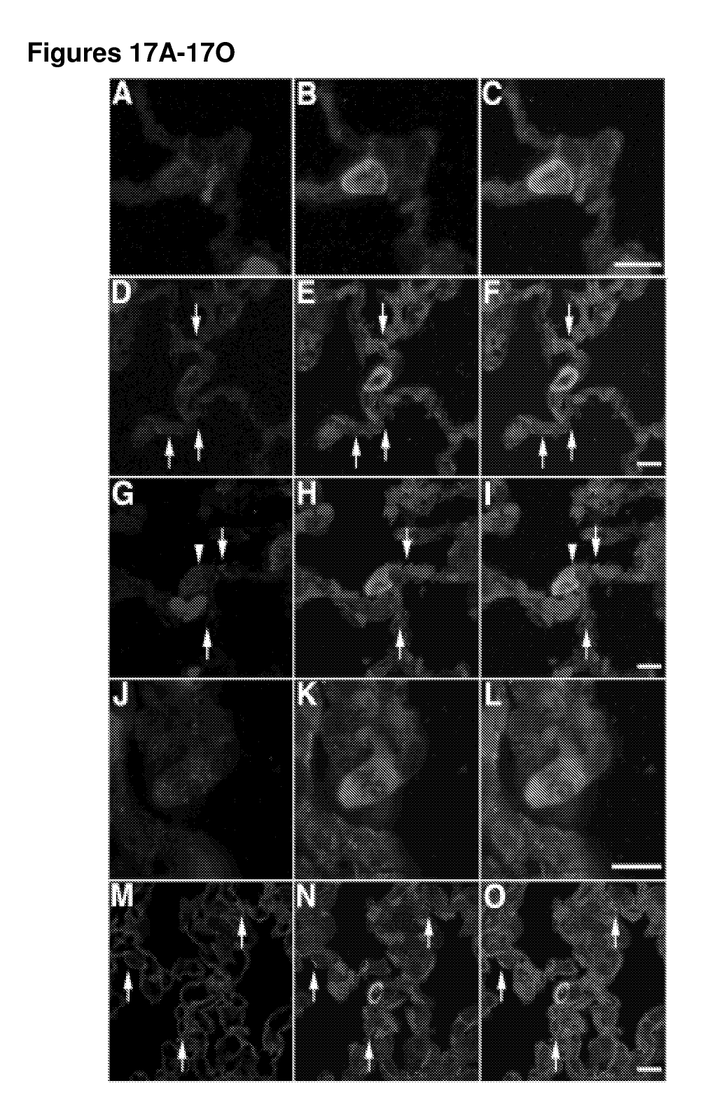

It was first investigated whether CB-CD34+ cells had the capacity to differentiate into alveolar type II cells, characterized by the presence of cytoplasmic immunoreactive surfactant-associated proteins. In lungs of Dox-treated single transgenic mice, only very rare (<1%) human-derived epithelial cells showed SP-C immunoreactivity (FIG. 15, FIGS. 17A-17C). In contrast, SP-C staining was easily detected in a significantly larger fraction of human-derived epithelial cells in lungs of double transgenic mice (FIG. 15, FIGS. 17D-17L). In both types of transgenic recipients, the intensity of SP-C immunoreactivity appeared lower in cord blood-derived surfactant-producing epithelial cells than in resident murine alveolar type II cells (FIGS. 17A-17L). Surfactant-positive granular material, consistent with surfactant-containing lamellar bodies, was often seen in close approximation to, or even protruding from the cell membrane, likely, without wishing to be limited or bound by theory, representing morphologic evidence of exocytosis (FIGS. 17G-17I

Examination of the parenchyma surrounding large-sized and ovoid or spherical cord blood-derived surfactant-containing type II-like cells revealed the frequent presence of more elongated cells containing small cytoplasmic aggregates of immunoreactive surfactant as well as human cytokeratin (FIGS. 17D-17I)These cells were highly indicative of so-called ‘transitional’ cells, which have phenotypical characteristics intermediate between type II cells (surfactant content) and type I cells (flat, elongated shape). The existence of cord blood-derived transitional cells indicates human-derived alveolar type II-like cells can be capable of generating alveolar type I cells, analogous to the function of native alveolar type II cells. In support of the potential progenitor capacity of the cord blood-derived surfactant-containing epithelial cells, mitotic activity was occasionally detected in cord blood-derived type II-like cells (FIGS. 17J-17L).

To further establish the potential of human cord blood-derived type II-like cells to generate alveolar type I cells, we assessed the presence of human-derived type I cells in the proximity of human-derived type II-like cells. Using the same double immunofluorescence approach, human cytokeratin labeling was combined with anti-T1alpha (podoplanin) staining (as marker of alveolar type I cells)⁶². Colocalization of immunoreactive human cytokeratin and T1alpha could be seen in cells surrounding cord blood-derived type II-like epithelial cells (FIGS. 17M-17O), indicating at least some of the type I cells surrounding cord blood-derived type II-like cells are human-derived as well.

Finally, we combined anti-human cytokeratin staining with anti-CCSP (Clara Cell Secretory Protein) labeling to determine possible generation of bronchial epithelial cells from cord blood stem cells. Colocalization of human cytokeratin and CCSP was not observed; this suggests differentiation of human CB-CD34+ cells to bronchial epithelial Clara cells was a rare event in this model.

Analysis of proliferation .To explore further the possibility of clonal expansion of cord blood-derived respiratory epithelial cells, we studied whether cord blood-derived cells were undergoing proliferation. As shown in FIGS. 17J-17L, mitotic activity was observed in cord blood-derived surfactant-producing type II cell-like epithelial cells. Proliferation of engrafted cord blood-derived cells was formally assessed by combining human alu-FISH analysis with anti-Ki67 immunofluorescence staining. Colocalization of Ki67- and human alu-FISH positivity to the same nuclei was readily observed in single as well as double transgenic recipients. Occasionally, clustering of Ki67-positive cord blood-derived cells was noted, indicative of clonal proliferation.

We also analyzed proliferation and cell fusion of engrafted CB-CD34+ cells at 8 weeks post-inoculation. A combination of anti-Ki67 immunofluorescence and alu FISH analysis showed two non-proliferating cord blood-derived cells, a proliferating cord blood-derived cell, and a proliferating murine cell in a single transgenic recipient. FISH analysis was performed using human alu-specific probes combined with anti-Ki67 immunofluorescence, DAPI counterstain. A combination of anti-Ki67 immunofluorescence and alu FISH analysis showed 4 Ki67-positive, proliferating cord blood-derived cells, three of which were in contiguity, suggestive of clonal expansion, in a double-transgenic recipient. Several proliferating murine nuclei (red) are noted. FISH analysis was performed using human alu-specific probes combined with anti-Ki67 immunofluorescence, DAPI counterstain. Combined anti-Ki67 and anti-human cytokeratin immunofluorescence showed a Ki67-positive, proliferating human-derived epithelial cell in a single transgenic recipient. Combined anti-Ki67 and anti-human cytokeratin immunofluorescence. showed a non-proliferating cord blood-derived epithelial cell, a proliferating cord blood-derived epithelial cell, and proliferating murine cells in a double transgenic recipient. Double FISH analysis was perfomed in single and double transgenic recipients using human alu-specific probes combined with mouse-specific pancentromeric probes. Murine FISH signal was absent in nuclei of human cord blood-derived cells. (FISH analysis was perfomed using human alu-specific probes combined with FISH analysis using mouse-specific pancentromeric probes, DAPI counterstain).

As shown in FIG. 16, the proliferative activity of cord blood-derived cells was significantly higher in double transgenic animals compared with single transgenic littermates, indicating proliferation of engrafted cells is promoted by lung injury. In both types of transgenic recipients, the proliferative activity was significantly higher in cord blood-derived cells than in native murine parenchymal cells.

To verify the proliferative potential of cord blood-derived epithelial cells, Ki67 labeling was also combined with human cytokeratin staining. Proliferative activity was readily observed in cord blood-derived cytokeratin-immunoreactive type II cell-like cells in single and double transgenic animals. These results demonstrate that human CB-CD34+ cells or their progeny, including respiratory epithelial cells, have the capacity for proliferation up to 8 weeks after intranasal inoculation.

Analysis of cell fusion. To determine whether cellular fusion of human and murine cells are implicated in the respiratory epithelial differentiation of CB-CD34+ cells observed in our model, we combined FISH analysis using human alu probes with FISH analysis using pan-centromeric murine chromosome probes. Color-coded dual FISH analysis allowed unequivocal differentiation between human and murine nuclei: human cord blood-derived nuclei were identified by diffuse intense green staining, whereas murine nuclei displayed a dot-like red staining pattern, corresponding to centromeric hybridization. Double FISH analysis using species-specific probes failed to reveal the presence of murine genomic material in numerous (>200) human-derived nuclei examined, indicating that fusion of human CB-CD34+ cells (or their progeny) with murine cells is not a dominant mechanism of generation of differentiated cord blood-derived cells in the model described herein.

### Discussion

In the studies described herein, it was demonstrated that human umbilical cord blood-derived CD34+ cells, delivered to newborn mice with injured lungs via intranasal inoculation, have the capacity to generate alveolar epithelial cells in vivo. Using state-of-the-art confocal microscopy techniques to circumvent the pitfalls of overlay and other imaging artifacts, it was further demonstrated that the human cord blood-derived alveolar epithelial cells have several critical phenotypic characteristics in common with resident alveolar epithelial type I and type II cells. Some cord blood-derived epithelial cells were relatively large-sized and cuboidal or spherical in shape, contained surfactant and were capable of replication, similar to native alveolar type II cells. Other cord blood-derived epithelial cells had an elongated shape and ovoid nuclei and contained membrane-associated immunoreactive podoplanin (T1alpha), which is a marker of alveolar type I cells⁶².

Based on the seminal work by Evans et al.⁶³ and Adamson and Bowden⁶⁴ more than three decades ago, the currently accepted paradigm of alveolar epithelial cell lineage and differentiation is that type II cells act as progenitor cells of the alveolar epithelium of the distal respiratory unit⁶⁵. Alveolar type II cells have the capacity to replicate and, by symmetric or asymmetric division, generate type II cells and/or type I cells. Type I cells, in contrast, are generally believed to be terminally differentiated and do not have the capacity to proliferate.

The coexistence of both type II cell-like and type I cell-like cord blood-derived epithelial cells, often in proximity to each other, indicate that the cord blood-derived type II-like cells are capable of assuming the function of progenitor of the terminal respiratory unit, analogous to the role of resident alveolar epithelial type II cells. The potential generation of type I cells from replicating cord blood-derived type II cells was supported by the abundant proliferative activity of cord blood-derived type II cell-like epithelial cells and the identification of cord blood-derived hybrid cells with a phenotype intermediate between type II and type I cells adjacent to cord blood-derived type II cell-like epithelial cells. Such transitional cells with characteristics of both type I and type II cells were first described at the ultrastructural level^{63,66,67}, where the existence of cells with the flattened shape of type I cells, combined with the irregular nucleus, microvilli and residual lamellar bodies of type II cells, was interpreted as evidence of the progenitor role of type II cells⁶⁴.

While surfactant immunoreactivity is routinely accepted as a marker of alveolar type II cells, the phenotypic characteristics of cord blood-derived surfactant-producing epithelial cells are being further investigated before these cells can be considered as transdifferentiated, mature alveolar type II cells. For instance, the functional characteristics of surfactant synthesis and secretion by the cord blood-derived surfactant-producing epithelial cells are being compared with those of native human alveolar type II cells. While exocytosis, and therefore the secretory machinery, appeared to be functional in cord blood-derived cell in the present study, their cytoplasmic surfactant content seemed to be lower than that of adjacent murine type II cells.

Engrafted cells were readily detected in single as well as double transgenic recipient animals. These results corroborate our previous observations with bone marrow-treated hyperoxic newborn mice²⁴ and indicate that the inherently high cell turnover of newborn lungs is sufficient to facilitate stem cell engraftment, as opposed to adult lungs in which cell injury appears to be a prerequisite for effective stem cell engraftment. The recovery rates of cord blood-derived cells at 8 weeks post-inoculation were similar in single and double transgenic animals. However, the proliferative activity of cord blood-derived cells at this time point was significantly higher in double transgenic animals.

The incidence of cord blood-derived surfactant-positive epithelial cells was significantly higher in double transgenic animals than in single transgenic littermates, indicating lung injury promoted phenotypic conversion of cord blood-derived CD34+ cells to alveolar epithelial cells. The exact mechanisms underlying injury-associated induction of proliferation and respiratory epithelial differentiation of CD34+ progenitor cells are being determined.

We also investigated the mechanisms underlying the generation of alveolar epithelial cells from human cord blood-derived CD34+ cells. Two major mechanisms, without wishing to be bound or limited by theory, have been suggested to account for the contribution of hematopoietic (bone marrow-derived or other) stem cells to adult tissue regeneration. One mechanism assumes a change in gene expression in response to the tissue micro-environment, a process referred to as transdifferentiation⁶⁸⁻⁷¹. According to the second proposed mechanism, changes in gene expression occur through fusion of hematopoietic stem cells with preexisting mature cells^{59,60,72}. Fusion of hematopoietic cells and tissue-specific host cells is a mechanism of generation of ‘transdifferentiated’ cells from bone marrow in the liver, brain, and heart^{59-61,72 73,74}.

Cell fusion in most systems is characterized by the coexistence of donor and recipient genomic material in the same nucleus⁵⁹⁻⁶¹. Our xenogeneic model allowed assessment of possible cell fusion by double FISH analysis using species-specific probes. Double FISH studies failed to reveal the presence of murine genomic material in human-derived nuclei, indicating the generation of human cord blood-derived epithelial cells is mediated exclusively or predominantly through fusion-independent mechanisms⁷⁵.

The studies described herein are the first to demonstrate that human cord blood-derived CD34+ cells are capable of reconstituting injured alveolar epithelium by stable and long-term engraftment, functional differentiation, replication and clonogenic expansion. These results have promising translational potential for the use of autologous or heterologous cord blood-derived CD34+ cells in a wide range of pulmonary diseases characterized by injured, deficient or defective respiratory epithelium, as described herein.

In other embodiments of the aspects described herein, the subpopulation of CD34+ cells most prone to undergo engraftment and secondary transdifferentiation to alveolar epithelial cells are enriched or isolated from the heterogeneous CD34+ cell population. In other embodiments of the aspects described herein, techniques to increase the initial graft size, focusing on the most relevant CD34+ cell subtype are performed. Such approaches to increase the CD34+ cell number include, but are not limited to, ex vivo expansion (e.g., preferably in culture conditions favoring subsequent engraftment and alveolar epithelial transdifferentiation) and/or combinations of multiple donor placentas. . In other embodiments of the aspects described herein, the engraftment and transdifferentiation potential of preterm—rather than term—CD34+ cells are determined.

In further embodiments of the aspects described herein, larger graft sizes are used to determine the effects of CB-CD34+ cells on lung growth kinetics and alveolarization.

The observations described herein offer the first solid evidence that cord blood-derived hematopoietic stem cells, delivered intratracheally, are capable of reconstituting injured alveolar epithelium. The demonstrated in vivo capacity of cord blood-derived hematopoietic progenitor cells to transdifferentiate into alveolar epithelial cells that display the surfactant production, replicative potential, and progenitor function characteristic of endogenous alveolar epithelial type II cells, demonstrates the use of cord blood-derived cells in regenerative pulmonary medicine. Knowledge acquired from the studies described herein in the developing lung are relevant for adult diseases characterized by alveolar injury, including acute respiratory distress syndrome (ARDS) and emphysema.

### REFERENCES

1. Jobe A H, Bancalari E: Bronchopulmonary dysplasia. Am J Respir Crit Care Med 2001, 163:1723-1729
2. Husain A N, Siddiqui N H, Stocker J T: Pathology of arrested acinar development in postsurfactant bronchopulmonary dysplasia. Hum Pathol 1998, 29:710-717
3. Jobe A J: The new BPD: an arrest of lung development. Pediatr Res 1999, 46:641-643
4. De Paepe ME, Mao Q, Powell J, Rubin S E, DeKoninck P, Appel N, Dixon M, Gundogan F: Growth of pulmonary microvasculature in ventilated preterm infants. Am J Respir Crit Care Med 2006, 173:204-211
5. Hargitai B, Szabo V, Hajdu J, Harmath A, Pataki M, Farid P, Papp Z, Szende B: Apoptosis in various organs of preterm infants: histopathologic study of lung, kidney, liver, and brain of ventilated infants. Pediatr Res 2001, 50:110-114
6. Lukkarinen H P, Laine J, Kaapa P O: Lung epithelial cells undergo apoptosis in neonatal respiratory distress syndrome. Pediatr Res 2003, 53:254-259
7. May M, Strobel P, Preisshofen T, Seidenspinner S, Marx A, Speer C P: Apoptosis and proliferation in lungs of ventilated and oxygen-treated preterm infants. Eur Respir J 2004, 23:113-121
8. De Paepe M E, Gundavarapu S, Tantravahi U, Pepperell J R, Haley S A, Luks F I, Mao Q: Fas-ligand-induced apoptosis of respiratory epithelial cells causes disruption of postcanalicular alveolar development. Am J Pathol 2008, 173:42-56
9. Sueblinvong V, Loi R, Eisenhauer P L, Bernstein I M, Suratt B T, Spees J L, Weiss D J: Derivation of lung epithelium from human cord blood-derived mesenchymal stem cells. Am J Respir Crit Care Med 2008, 177:701-711
10. Macpherson H, Keir P, Webb S, Samuel K, Boyle S, Bickmore W, Forrester L, Dorin J: Bone marrow-derived SP cells can contribute to the respiratory tract of mice in vivo. J Cell Sci 2005, 118:2441-2450
11. MacPherson H, Keir P A, Edwards C J, Webb S, Dorin J R: Following damage, the majority of bone marrow-derived airway cells express an epithelial marker. Respir Res 2006, 7:145
12. Loi R, Beckett T, Goncz K K, Suratt B T, Weiss D J: Limited restoration of cystic fibrosis lung epithelium in vivo with adult bone marrow-derived cells. Am J Respir Crit Care Med 2006, 173:171-179
13. Yan C, Lian X, Dai Y, Wang X, Qu P, White A, Qin Y, Du H: Gene delivery by the hSP-B promoter to lung alveolar type II epithelial cells in LAL-knockout mice through bone marrow mesenchymal stem cells. Gene Ther 2007, 14:1461-1470
14. Serikov V B, Popov B, Mikhailov V M, Gupta N, Matthay M A: Evidence of temporary airway epithelial repopulation and rare clonal formation by BM-derived cells following naphthalene injury in mice. Anat Rec (Hoboken) 2007, 290:1033-1045
15. Wong A P, Dutly A E, Sacher A, Lee H, Hwang D M, Liu M, Keshavjee S, Hu J, Waddell T K: Targeted cell replacement with bone marrow cells for airway epithelial regeneration. Am J Physiol Lung Cell Mol Physiol 2007, 293:L740-752
16. Bruscia E M, Price J E, Cheng E C, Weiner S, Caputo C, Ferreira E C, Egan M E, Krause D S: Assessment of cystic fibrosis transmembrane conductance regulator (CFTR) activity in CFTR-null mice after bone marrow transplantation. Proc Natl Acad Sci U S A 2006, 103:2965-2970
17. Bruscia E M, Ziegler E C, Price J E, Weiner S, Egan M E, Krause D S: Engraftment of donor-derived epithelial cells in multiple organs following bone marrow transplantation into newborn mice. Stem Cells 2006, 24:2299-2308
18. Spees J L, Pociask D A, Sullivan D E, Whitney M J, Lasky J A, Prockop D J, Brody A R: Engraftment of bone marrow progenitor cells in a rat model of asbestos-induced pulmonary fibrosis. Am J Respir Crit Care Med 2007, 176:385-394
19. Raoul W, Wagner-Ballon O, Saber G, Hulin A, Marcos E, Giraudier S, Vainchenker W, Adnot S, Eddahibi S, Maitre B: Effects of bone marrow-derived cells on monocrotaline- and hypoxia-induced pulmonary hypertension in mice. Respir Res 2007, 8:8
20. Weiss D J, Kolls JK, Ortiz L A, Panoskaltsis-Mortari A, Prockop D J: Stem cells and cell therapies in lung biology and lung diseases. Proc Am Thorac Soc 2008, 5:637-667
21. Kotton D N, Fabian A J, Mulligan R C: Failure of bone marrow to reconstitute lung epithelium. Am J Respir Cell Mol Biol 2005, 33:328-334
22. Wagers A J, Sherwood R I, Christensen J L, Weissman I L: Little evidence for developmental plasticity of adult hematopoietic stem cells. Science 2002, 297:2256-2259
23. Chang JC, Summer R, Sun X, Fitzsimmons K, Fine A: Evidence that bone marrow cells do not contribute to the alveolar epithelium. Am J Respir Cell Mol Biol 2005, 33:335-342
24. Fritzell J A, Jr., Mao Q, Gundavarapu S, Pasquariello T, Aliotta J M, Ayala A, Padbury J F, De Paepe ME: Fate and effects of adult bone marrow cells in lungs of normoxic and hyperoxic newborn mice. Am J Respir Cell Mol Biol 2009, 40:575-587
25. Rojas M, Xu J, Woods C R, Mora A L, Spears W, Roman J, Brigham K L: Bone marrow-derived mesenchymal stem cells in repair of the injured lung. Am J Respir Cell Mol Biol 2005, 33:145-152
26. Spees J L, Whitney M J, Sullivan D E, Lasky J A, Laboy M, Ylostalo J, Prockop D J: Bone marrow progenitor cells contribute to repair and remodeling of the lung and heart in a rat model of progressive pulmonary hypertension. Faseb J 2008, 22:1226-1236
27. Aliotta J M, Keaney P, Passero M, Dooner M S, Pimentel J, Greer D, Demers D, Foster B, Peterson A, Dooner G, Theise N D, Abedi M, Colvin G A, Quesenberry P J: Bone marrow production of lung cells: the impact of G-CSF, cardiotoxin, graded doses of irradiation, and subpopulation phenotype. Exp Hematol 2006, 34:230-241
28. Nakahata T, Ogawa M: Hemopoietic colony-forming cells in umbilical cord blood with extensive capability to generate mono- and multipotential hemopoietic progenitors. J Clin Invest 1982, 70:1324-1328
29. Broxmeyer H E, Douglas G W, Hangoc G, Cooper S, Bard J, English D, Amy M, Thomas L, Boyse E A: Human umbilical cord blood as a potential source of transplantable hematopoietic stem/progenitor cells. Proc Natl Acad Sci U S A 1989, 86:3828-3832
30. Erices A, Conget P, Minguell J J: Mesenchymal progenitor cells in human umbilical cord blood. Br J Haematol 2000, 109:235-242
31. Berger M J, Adams S D, Tigges B M, Sprague S L, Wang X J, Collins D P, McKenna D H: Differentiation of umbilical cord blood-derived multilineage progenitor cells into respiratory epithelial cells. Cytotherapy 2006, 8:480-487
32. Kim J W, Kim S Y, Park S Y, Kim Y M, Kim J M, Lee M H, Ryu H M: Mesenchymal progenitor cells in the human umbilical cord. Ann Hematol 2004, 83:733-738
33. Goldberg J L, Laughlin M J, Pompili V J: Umbilical cord blood stem cells: implications for cardiovascular regenerative medicine. J Mol Cell Cardiol 2007, 42:912-920
34. van de Ven C, Collins D, Bradley M B, Morris E, Cairo M S: The potential of umbilical cord blood multipotent stem cells for nonhematopoietic tissue and cell regeneration. Exp Hematol 2007, 35:1753-1765
35. Garbuzova-Davis S, Willing A E, Zigova T, Saporta S, Justen E B, Lane J C, Hudson J E, Chen N, Davis CD, Sanberg P R: Intravenous administration of human umbilical cord blood cells in a mouse model of amyotrophic lateral sclerosis: distribution, migration, and differentiation. J Hematother Stem Cell Res 2003, 12:255-270
36. Dai Y, Li J, Dai G, Mu H, Wu Q, Hu K, Cao Q: Skin epithelial cells in mice from umbilical cord blood mesenchymal stem cells. Burns 2007, 33:418-428
37. Harris D T, Badowski M, Ahmad N, Gaballa M A: The potential of cord blood stem cells for use in regenerative medicine. Expert Opin Biol Ther 2007, 7:1311-1322
38. Almeida-Porada G, Porada C D, Chamberlain J, Torabi A, Zanjani E D: Formation of human hepatocytes by human hematopoietic stem cells in sheep. Blood 2004, 104:2582-2590
39. Hirata Y, Sata M, Motomura N, Takanashi M, Suematsu Y, Ono M, Takamoto S: Human umbilical cord blood cells improve cardiac function after myocardial infarction. Biochem Biophys Res Commun 2005, 327:609-614
40. Sharma A D, Cantz T, Richter R, Eckert K, Henschler R, Wilkens L, Jochheim-Richter A, Arseniev L, Ott M: Human cord blood stem cells generate human cytokeratin 18-negative hepatocyte-like cells in injured mouse liver. Am J Pathol 2005, 167:555-564
41. Wang T T, Tio M, Lee W, Beerheide W, Udolph G: Neural differentiation of mesenchymal-like stem cells from cord blood is mediated by PKA. Biochem Biophys Res Commun 2007, 357:1021-1027
42. Di Campli C, Piscaglia A C, Rutella S, Bonanno G, Vecchio F M, Zocco M A, Monego G, Michetti F, Mancuso S, Pola P, Leone G, Gasbarrini G, Gasbarrini A: Improvement of mortality rate and decrease in histologic hepatic injury after human cord blood stem cell infusion in a murine model of hepatotoxicity. Transplant Proc 2005, 37:2707-2710
43. Ishikawa F, Yasukawa M, Yoshida S, Nakamura K, Nagatoshi Y, Kanemaru T, Shimoda K, Shimoda S, Miyamoto T, Okamura J, Shultz L D, Harada M: Human cord blood- and bone marrow-derived CD34+ cells regenerate gastrointestinal epithelial cells. Faseb J 2004, 18:1958-1960
44. Yoshida S, Ishikawa F, Kawano N, Shimoda K, Nagafuchi S, Shimoda S, Yasukawa M, Kanemaru T, Ishibashi H, Shultz L D, Harada M: Human cord blood--derived cells generate insulin-producing cells in vivo. Stem Cells 2005, 23:1409-1416
45. Chang Y S, Oh W, Choi S J, Sung D K, Kim S Y, Choi E Y, Kang S, Jin H J, Yang Y S, Park W S: Human umbilical cord blood-derived mesenchymal stem cells attenuate hyperoxia-induced lung injury in neonatal rats. Cell Transplant 2009, 18:869-886
46. Moodley Y, Atienza D, Manuelpillai U, Samuel CS, Tchongue J, Ilancheran S, Boyd R, Trounson A: Human umbilical cord mesenchymal stem cells reduce fibrosis of bleomycin-induced lung injury. Am J Pathol 2009, 175:303-313
47. Brody A R, Salazar K D, Lankford S M: Mesenchymal stem cells modulate lung injury. Proc Am Thorac Soc 2010, 7:130-133
48. Tolar J, Nauta A J, Osborn M J, Panoskaltsis Mortari A, McElmurry R T, Bell S, Xia L, Zhou N, Riddle M, Schroeder T M, Westendorf J J, Mclvor R S, Hogendoorn P C, Szuhai K, Oseth L, Hirsch B, Yant S R, Kay M A, Peister A, Prockop D J, Fibbe W E, Blazar B R: Sarcoma derived from cultured mesenchymal stem cells. Stem Cells 2007, 25:371-379
49. Wang Y, Huso D L, Harrington J, Kellner J, Jeong D K, Turney J, McNiece I K: Outgrowth of a transformed cell population derived from normal human BM mesenchymal stem cell culture. Cytotherapy 2005, 7:509-519
50. Aguilar S, Nye E, Chan J, Loebinger M, Spencer-Dene B, Fisk N, Stamp G, Bonnet D, Janes S M: Murine but not human mesenchymal stem cells generate osteosarcoma-like lesions in the lung. Stem Cells 2007, 25:1586-1594
51. De Paepe M E, Haley S A, Lacourse Z, Mao Q: Effects of Fas-ligand overexpression on alveolar type II cell growth kinetics in perinatal murine lungs. Pediatr Res 2010, 68:57-62
52. Lee R J, Fang Q, Davol P A, Gu Y, Sievers R E, Grabert R C, Gall J M, Tsang E, Yee M S, Fok H, Huang N F, Padbury J F, Larrick J W, Lum L G: Antibody targeting of stem cells to infarcted myocardium. Stem Cells 2007, 25:712-717
53. Zhao T C, Tseng A, Yano N, Tseng Y, Davol P A, Lee R J, Lum L G, Padbury J F: Targeting human CD34+ hematopoietic stem cells with anti-CD45× anti-myosin light-chain bispecific antibody preserves cardiac function in myocardial infarction. J Appl Physiol 2008, 104:1793-1800
54. Tichelaar J W, Lu W, Whitsett J A: Conditional expression of fibroblast growth factor-7 in the developing and mature lung. J Biol Chem 2000, 275:11858-11864
55. McBride C, Gaupp D, Phinney D G: Quantifying levels of transplanted murine and human mesenchymal stem cells in vivo by real-time PCR. Cytotherapy 2003, 5:7-18
56. Schormann W, Hammersen F J, Brulport M, Hermes M, Bauer A, Rudolph C, Schug M, Lehmann T, Nussler A, Ungefroren H, Hutchinson J, Fandrich F, Petersen J, Wursthorn K, Burda M R, Brustle O, Krishnamurthi K, von Mach M, Hengstler J G: Tracking of human cells in mice. Histochem Cell Biol 2008, 130:329-338
57. Farr A G, Berry M L, Kim A, Nelson A J, Welch M P, Aruffo A: Characterization and cloning of a novel glycoprotein expressed by stromal cells in T-dependent areas of peripheral lymphoid tissues. J Exp Med 1992, 176:1477-1482
58. Kotton D N, Ma B Y, Cardoso W V, Sanderson E A, Summer R S, Williams M C, Fine A: Bone marrow-derived cells as progenitors of lung alveolar epithelium. Development 2001, 128:5181-5188
59. Terada N, Hamazaki T, Oka M, Hold M, Mastalerz D M, Nakano Y, Meyer E M, Morel L, Petersen B E, Scott E W: Bone marrow cells adopt the phenotype of other cells by spontaneous cell fusion. Nature 2002, 416:542-545
60. Vassilopoulos G, Wang P R, Russell D W: Transplanted bone marrow regenerates liver by cell fusion. Nature 2003, 422:901-904
61. Alvarez-Dolado M, Pardal R, Garcia-Verdugo J M, Fike J R, Lee H O, Pfeffer K, Lois C, Morrison SJ, Alvarez-Buylla A: Fusion of bone-marrow-derived cells with Purkinje neurons, cardiomyocytes and hepatocytes. Nature 2003, 425:968-973
62. Barth K, Blasche R, Kasper M: T1alpha/podoplanin shows raft-associated distribution in mouse lung alveolar epithelial E10 cells. Cell Physiol Biochem 2010, 25:103-112
63. Evans M J, Cabral L J, Stephens R J, Freeman G: Transformation of alveolar type 2 cells to type 1 cells following exposure to NO2. Exp Mol Pathol 1975, 22:142-150
64. Adamson I Y, Bowden D H: The type 2 cell as progenitor of alveolar epithelial regeneration. A cytodynamic study in mice after exposure to oxygen. Lab Invest 1974, 30:35-42
65. Dobbs L G, Johnson M D, Vanderbilt J, Allen L, Gonzalez R: The great big alveolar TI cell: evolving concepts and paradigms. Cell Physiol Biochem 2010, 25:55-62
66. Faulkner C S, 2nd, Esterly J R: Ultrastructural changes in the alveolar epithelium in response to Freund's adjuvant. Am J Pathol 1971, 64:559-566
67. Evans M J, Cabral L C, Stephens R J, Freeman G: Acute kinetic response and renewal of the alveolar epithelium following injury by nitrogen dioxide. Chest 1974, 65:Suppl:62S-655
68. LaBarge M A, Blau H M: Biological progression from adult bone marrow to mononucleate muscle stem cell to multinucleate muscle fiber in response to injury. Cell 2002, 111:589-601
69. Lagasse E, Connors H, Al-Dhalimy M, Reitsma M, Dohse M, Osborne L, Wang X, Finegold M, Weissman I L, Grompe M: Purified hematopoietic stem cells can differentiate into hepatocytes in vivo. Nat Med 2000, 6:1229-1234
70. Krause D S, Theise N D, Collector M I, Henegariu O, Hwang S, Gardner R, Neutzel S, Sharkis S J: Multi-organ, multi-lineage engraftment by a single bone marrow-derived stem cell. Cell 2001, 105:369-377
71. Mezey E, Chandross K J, Harta G, Maki R A, McKercher S R: Turning blood into brain: cells bearing neuronal antigens generated in vivo from bone marrow. Science 2000, 290:1779-1782
72. Wang X, Willenbring H, Akkari Y, Torimaru Y, Foster M, Al-Dhalimy M, Lagasse E, Finegold M, Olson S, Grompe M: Cell fusion is the principal source of bone-marrow-derived hepatocytes. Nature 2003, 422:897-901
73. Weimann J M, Johansson C B, Trejo A, Blau H M: Stable reprogrammed heterokaryons form spontaneously in Purkinje neurons after bone marrow transplant. Nat Cell Biol 2003, 5:959-966
74. Ying Q L, Nichols J, Evans E P, Smith A G: Changing potency by spontaneous fusion. Nature 2002, 416:545-548
75. Aliotta J M, Sanchez-Guijo F M, Dooner G J, Johnson K W, Dooner M S, Greer K A, Greer D, Pimentel J, Kolankiewicz L M, Puente N, Faradyan S, Ferland P, Bearer E L, Passero M A, Adedi M, Colvin G A, Quesenberry P J: Alteration of marrow cell gene expression, protein production, and engraftment into lung by lung-derived microvesicles: a novel mechanism for phenotype modulation. Stem Cells 2007, 25:2245-2256
76. Fischer N, Leger O: Bispecific antibodies: molecules that enable novel therapeutic strategies. Pathobiology 2007, 74:3-14
77. Zhao T C, Tseng A, Yano N, Tseng Y, Davol P A, Lee R J, Lum L G, Padbury J F: Targeting human CD34+ hematopoietic stem cells with anti-CD45× anti-myosin light-chain bispecific antibody preserves cardiac function in myocardial infarction. J Appl Physiol 2008, 104:1793-1800
78. Lum L G, Fok H, Sievers R, Abedi M, Quesenberry P J, Lee R J: Targeting of Lin-Sca+ hematopoietic stem cells with bispecific antibodies to injured myocardium. Blood Cells Mol Dis 2004, 32:82-87
79. Fritzell J A, Jr., Mao Q, Gundavarapu S, Pasquariello T, Aliotta J M, Ayala A, Padbury J F, De Paepe M E: Fate and effects of adult bone marrow cells in lungs of normoxic and hyperoxic newborn mice. Am J Respir Cell Mol Biol 2009, 40:575-587
80. Lapidot T: Mechanism of human stem cell migration and repopulation of NOD/SCID and B2mnull NOD/SCID mice. The role of SDF-1/CXCR4 interactions. Ann N Y Acad Sci 2001, 938:83-95
81. Lapidot T, Kollet O: The essential roles of the chemokine SDF-1 and its receptor CXCR4 in human stem cell homing and repopulation of transplanted immune-deficient NOD/SCID and NOD/SCID/B2m(null) mice. Leukemia 2002, 16:1992-2003
82. Sueblinvong V, Goodwin M A, Eisenhauer P L, Bernstein I M, Spees J L, Weiss D J: Cord Blood-derived Mesenchymal Stem Cells Can Participate in Lung Epithelial Remodeling In vivo. Proc Am Thorac Soc 2008, 5:362
83. De Paepe M E, Gundavarapu S, Tantravahi U, Pepperell J R, Haley S A, Luks F I, Mao Q: Fas-ligand-induced apoptosis of respiratory epithelial cells causes disruption of postcanalicular alveolar development. Am J Pathol 2008, 173:42-56

1. A method for treating or preventing a lung disorder in a subject in need thereof, comprising administering a therapeutically effective amount of a population of isolated or enriched umbilical cord blood derived hematopoietic stem cells to said subject.

2. A method for repairing or reconstituting or generating pulmonary epithelium in a subject in need thereof, comprising administering a population of isolated or enriched umbilical cord blood derived hematopoietic stem cells to said subject.

3. A method for repairing or reconstituting or generating pulmonary vasculature or pulmonary endothelium in a subject in need thereof, comprising administering a population of isolated or enriched umbilical cord blood derived hematopoietic stem cells to said subject.

4. A method for repairing or reconstituting pulmonary alveoli in a subject in need thereof, comprising administering a population of isolated or enriched umbilical cord blood derived hematopoietic stem cells to said subject.

5. The method of claim 1, further comprising selecting a subject who is suffering from a lung disorder prior to administering the population of isolated or enriched umbilical cord blood derived hematopoietic stem cells.

6. The method of claim 2, further comprising selecting a subject in need of repair or reconstitution or generation of pulmonary epithelium prior to administering the population of isolated or enriched umbilical cord blood derived hematopoietic stem cells.

7. The method of claim 3, further comprising selecting a subject in need of repair or reconstitution or generation of pulmonary vasculature or pulmonary endothelium prior to administering the population of isolated or enriched umbilical cord blood derived hematopoietic stem cells.

8. The method of claim 4, further comprising selecting a subject in need of repair or reconstitution or regeneration of pulmonary alveoli prior to administering the population of isolated or enriched umbilical cord blood derived hematopoietic stem cells.

9. The method of claim 2, wherein the administration is intrapulmonary administration, systemic administration, or any combination thereof.

10. The method of claim 9, wherein the intrapulmonary administration is intratreacheal or intranasal administration.

11. (canceled)

12. (canceled)

13. (canceled)

14. The method of claim 2, wherein the population of isolated or enriched umbilical cord blood derived hematopoietic stem cells are expanded ex vivo prior to administration to the subject.

15. The method of claim 2, wherein the hematopoietic stem cells are selected based on positive expression of CD34.

16. The method of claim 2, wherein the subject is an intubated subject.

17. The method of claim 2, wherein the subject is an infant or preterm infant.

18. The method of claim 2, wherein the population of isolated or enriched umbilical cord blood derived hematopoietic stem cells are autologous cells.

19. The method of claim 2, wherein the population of isolated or enriched umbilical cord blood derived hematopoietic stem cells are allogeneic cells obtained from one or more donors.

20. The method of claim 2, further comprising administering at least one therapeutic agent.

21. The method of claim 2, further comprising arming the population of isolated or enriched umbilical cord blood derived hematopoietic stem cells with at least one therapeutic agent.

22. The method of claim 20, wherein the at least one therapeutic agent enhances homing, engraftment, or survival of the population of isolated or enriched umbilical cord blood derived hematopoietic stem cells.

23. The method of claim 20, wherein the at least one therapeutic agent comprises a bispecific antibody.

24. The method of claim 23, wherein the bispecific antibody is an antibody specific for CD34 and VCAM-1.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
